SEQUENCE LISTING (78 sequences)

SEQ ID NO: 1 - DNA, 35 bp, Homo sapiens
ctggagctggagaaaaaaaaaactcctctacatac

SEQ ID NO: 2 - DNA, 35 bp, Homo sapiens
ctggagctggaggttattgtttgggttaataaatg

SEQ ID NO: 3 - DNA, 21 bp, Homo sapiens
  misc_feature (21)..(21): Fluoresceine label
ttttttttttcggacgtcgtt

SEQ ID NO: 4 - DNA, 20 bp, Homo sapiens
  misc_feature (1)..(1): red640, a LightCycler fluorescence label for channel F2
tcggtcgatgttttcggtaa

SEQ ID NO: 5 - DNA, 110 bp, Homo sapiens
aaaaaaaaaactcctctacatacgccgcgaataaattaccgaaaacatcgaccgaacaacgacgtccgaaaaaaaaaaaacgaactccatttattaacccaaacaataac

SEQ ID NO: 6 - DNA, 26 bp, Homo sapiens
  misc_feature (7)..(26): n stands for a, c, g, or t
tccracnnnnnnnnnnnnnnnnnnnn

SEQ ID NO: 7 - DNA, 26 bp, Homo sapiens
  misc_feature (7)..(26): n stands for a, c, g, or t
aaggagnnnnnnnnnnnnnnnnnnnn

SEQ ID NO: 8 - DNA, 22 bp, Homo sapiens
  misc_feature (7)..(22): n stands for a, c, g or t
ctggagnnnnnnnnnnnnnnnn

SEQ ID NO: 9 - DNA, 22 bp, Homo sapiens
  misc_feature (7)..(22): n stands for a, c, g or t
ctgragnnnnnnnnnnnnnnnn

SEQ ID NO: 10 - DNA, 22 bp, Homo sapiens
  misc_feature (7)..(22): n stands for a, c, g or t
ctgaagnnnnnnnnnnnnnnnn

SEQ ID NO: 11 - DNA, 22 bp, Homo sapiens
  misc_feature (7)..(22): n stands for a, c, g or t
gtgcagnnnnnnnnnnnnnnnn

SEQ ID NO: 12 - DNA, 22 bp, Homo sapiens
  misc_feature (7)..(22): n stands for a, c, g or t
cttgagnnnnnnnnnnnnnnnn

SEQ ID NO: 13 - DNA, 26 bp, Homo sapiens
  misc_feature (4)..(9): n stands for a, c, g or t
ccannnnnngtnnnnnnnnnnnnnnn

SEQ ID NO: 14 - DNA, 26 bp, Homo sapiens
  misc_feature (6)..(26): n stands for a, c, g or t
grtacnnnngtnnnnnnnnnnnnnnn

SEQ ID NO: 15 - DNA, 25 bp, Homo sapiens
  misc_feature (4)..(25): n stands for a, c, g or t
gaynnnnnrtcnnnnnnnnnnnnnn

SEQ ID NO: 16 - DNA, 26 bp, Homo sapiens
  misc_feature (4)..(26): n stands for a, c, g or t
ccannnnnnntcnnnnnnnnnnnnnn

SEQ ID NO: 17 - DNA, 25 bp, Homo sapiens
  misc_feature (3)..(25): n stands for a, c, g or t
acnnnnnntggnnnnnnnnnnnnnn

SEQ ID NO: 18 - DNA, 25 bp, Homo sapiens
  misc_feature (4)..(25): n stands for a, c, g or t
ggannnnnngtgnnnnnnnnnnnnn

SEQ ID NO: 19 - DNA, 25 bp, Homo sapiens
  misc_feature (5)..(25): n stands for a, c, g or t
gaacnnnnnctcnnnnnnnnnnnnn

SEQ ID NO: 20 - DNA, 24 bp, Homo sapiens
  misc_feature (4)..(24)
  misc_feature (4)..(24): n stands for a, c, g or t
gabnnnnnrtcnnnnnnnnnnnnn

SEQ ID NO: 21 - DNA, 24 bp, Homo sapiens
  misc_feature (4)..(24): n stands for a, c, g or t
gaynnnnnvtcnnnnnnnnnnnnn

SEQ ID NO: 22 - DNA, 24 bp, Homo sapiens
  misc_feature (4)..(24): n stands for a, c, g or t
gaynnnnnrtcnnnnnnnnnnnnn

SEQ ID NO: 23 - DNA, 24 bp, Homo sapiens
  misc_feature (4)..(24): n stands for a, c, g or t
aagnnnnncttnnnnnnnnnnnnn

SEQ ID NO: 24 - DNA, 25 bp, Homo sapiens
  misc_feature (5)..(25): n stands for a, c, g or t
ccacnnnnnttgnnnnnnnnnnnnn

SEQ ID NO: 25 - DNA, 25 bp, Homo sapiens
  misc_feature (3)..(25): n stands for a, c, g or t
gannnnnnntggnnnnnnnnnnnnn

SEQ ID NO: 26 - DNA, 25 bp, Homo sapiens
  misc_feature (4)..(25): n stands for a, c, g or t
ccannnnnngtcnnnnnnnnnnnnn

SEQ ID NO: 27 - DNA, 24 bp, Homo sapiens
  misc_feature (4)..(24): n stands for a, c, g or t
gagnnnnnctcnnnnnnnnnnnnn

SEQ ID NO: 28 - DNA, 24 bp, Homo sapiens
  misc_feature (4)..(24): n stands for a, c, g or t
cacnnnnnntccnnnnnnnnnnnn

SEQ ID NO: 29 - DNA, 18 bp, Homo sapiens
  misc_feature (7)..(18): n stands for a, c, g or t
cccacannnnnnnnnnnn

SEQ ID NO: 30 - DNA, 25 bp, Homo sapiens
  misc_feature (4)..(25): n stands for a, c, g or t
gtannnnnngttcnnnnnnnnnnnn

SEQ ID NO: 31 - DNA, 25 bp, Homo sapiens
  misc_feature (5)..(25): n stands for a, c, g or t
gaacnnnnnntacnnnnnnnnnnnn

SEQ ID NO: 32 - DNA, 24 bp, Homo sapiens
  misc_feature (4)..(24): n stands for a, c, g or t
gagnnnnngttcnnnnnnnnnnnn

SEQ ID NO: 33 - DNA, 24 bp, Homo sapiens
  misc_feature (4)..(24): n stands for a, c, g or t
caannnnngtggnnnnnnnnnnnn

SEQ ID NO: 34 - DNA, 24 bp, Homo sapiens
  misc_feature (4)..(24): n stands for a, c, g or t
gacnnnnnntggnnnnnnnnnnnn

SEQ ID NO: 35 - DNA, 23 bp, Homo sapiens
  misc_feature (5)..(23): n stands for a, c, g or t
ggagnnnnngtnnnnnnnnnnnn

SEQ ID NO: 36 - DNA, 24 bp, Homo sapiens
  misc_feature (4)..(24): n stands for a, c, g or t
tgannnnnntcannnnnnnnnnnn

SEQ ID NO: 37 - DNA, 24 bp, Homo sapiens
  misc_feature (4)..(24): n stands for a, c, g or t
gcannnnnntcgnnnnnnnnnnnn

SEQ ID NO: 38 - DNA, 24 bp, Homo sapiens
  misc_feature (4)..(24): n stands for a, c, g or t
cgannnnnntgcnnnnnnnnnnnn

SEQ ID NO: 39 - DNA, 17 bp, Homo sapiens
  misc_feature (6)..(17): n stands for a, c, g or t
acggcnnnnnnnnnnnn

SEQ ID NO: 40 - DNA, 17 bp, Homo sapiens
  misc_feature (6)..(17): n stands for a, c, g or t
acggcnnnnnnnnnnnn

SEQ ID NO: 41 - DNA, 23 bp, Homo sapiens
  misc_feature (3)..(23): n stands for a, c, g or t
acnnnngtaycnnnnnnnnnnnn

SEQ ID NO: 42 - DNA, 25 bp, Homo sapiens
  misc_feature (4)..(25): n stands for a, c, g or t
ggannnnnngttcnnnnnnnnnnnn

SEQ ID NO: 43 - DNA, 25 bp, Homo sapiens
  misc_feature (5)..(25): n stands for a, c, g or t
gaacnnnnnntccnnnnnnnnnnnn

SEQ ID NO: 44 - DNA, 24 bp, Homo sapiens
  misc_feature (4)..(24): n stands for a, c, g or t
gcannnnnntgcnnnnnnnnnnnn

SEQ ID NO: 45 - DNA, 24 bp, Homo sapiens
  misc_feature (4)..(24): n stands for a, c, g or t
gcannnnnntgcnnnnnnnnnnnn

SEQ ID NO: 46 - DNA, 26 bp, Homo sapiens
  misc_feature (5)..(26): n stands for a, c, g or t
ccaannnnnnnttcnnnnnnnnnnnn

SEQ ID NO: 47 - DNA, 17 bp, Homo sapiens
  misc_feature (7)..(17): n stands for a, c, g or t
caarcannnnnnnnnnn

SEQ ID NO: 48 - DNA, 16 bp, Homo sapiens
  misc_feature (6)..(16): n stands for a, c, g or t
acggannnnnnnnnnn

SEQ ID NO: 49 - DNA, 16 bp, Homo sapiens
  misc_feature (6)..(16): n stands for a, c, g or t
atgaannnnnnnnnnn

SEQ ID NO: 50 - DNA, 17 bp, Homo sapiens
  misc_feature (7)..(17): n stands for a, c, g or t
tarccannnnnnnnnnn

SEQ ID NO: 51 - DNA, 17 bp, Homo sapiens
  misc_feature (7)..(17): n stands for a, c, g or t
cacccannnnnnnnnnn

SEQ ID NO: 52 - DNA, 17 bp, Homo sapiens
  misc_feature (7)..(17): n stands for a, c, g or t
gaccgannnnnnnnnnn

SEQ ID NO: 53 - DNA, 17 bp, Homo sapiens
  misc_feature (7)..(17): n stands for a, c, g or t
ggcggannnnnnnnnnn

SEQ ID NO: 54 - DNA, 25 bp, Homo sapiens
  misc_feature (4)..(25): n stands for a, c, g or t
gaannnnnnnttggnnnnnnnnnnn

SEQ ID NO: 55 - DNA, 15 bp, Homo sapiens
  misc_feature (6)..(15): n stands for a, c, g or t
ggatgnnnnnnnnnn

SEQ ID NO: 56 - DNA, 16 bp, Homo sapiens
  misc_feature (7)..(16): n stands for a, c, g or t
gcgatgnnnnnnnnnn

SEQ ID NO: 57 - DNA, 15 bp, Homo sapiens
  misc_feature (6)..(15): n stands for a, c, g or t
gggacnnnnnnnnnn

SEQ ID NO: 58 - DNA, 16 bp, Homo sapiens
  misc_feature (7)..(16): n stands for a, c, g or t
gaggagnnnnnnnnnn

SEQ ID NO: 59 - DNA, 15 bp, Homo sapiens
  misc_feature (6)..(15): n stands for a, c, g or t
ctcagnnnnnnnnnn

SEQ ID NO: 60 - DNA, 21 bp, Homo sapiens
  misc_feature (3)..(21): n stands for a, c, g or t
acnnnnnctccnnnnnnnnnn

SEQ ID NO: 61 - DNA, 14 bp, Homo sapiens
  misc_feature (6)..(14): n stands for a, c, g or t
ggatgnnnnnnnnn

SEQ ID NO: 62 - DNA, 14 bp, Homo sapiens
  misc_feature (6)..(14): n stands for a, c, g or t
ctcagnnnnnnnnn

SEQ ID NO: 63 - DNA, 13 bp, Homo sapiens
  misc_feature (6)..(13): n stands for a, c, g or t
ggtgannnnnnnn

SEQ ID NO: 64 - DNA, 13 bp, Homo sapiens
  misc_feature (6)..(13): n stands for a, c, g or t
gaagannnnnnnn

SEQ ID NO: 65 - DNA, 13 bp, Homo sapiens
  misc_feature (6)..(13): n stands for a, c, g or t
ggtgannnnnnnn

SEQ ID NO: 66 - DNA, 13 bp, Homo sapiens
  misc_feature (6)..(13): n stands for a, c, g or t
gcagcnnnnnnnn

SEQ ID NO: 67 - DNA, 11 bp, Homo sapiens
  misc_feature (5)..(11): n stands for a, c, g or t
cctcnnnnnnn

SEQ ID NO: 68 - DNA, 13 bp, Homo sapiens
  misc_feature (7)..(13): n stands for a, c, g or t
gaagacnnnnnnn

SEQ ID NO: 69 - DNA, 13 bp, Homo sapiens
  misc_feature (7)..(13): n stands for a, c, g or t
cagctcnnnnnnn

SEQ ID NO: 70 - DNA, 35 bp, Artificial sequence; identical to SEQ ID NO: 1
  Oligonunucleotides

SEQ ID NO: 71 - DNA, 35 bp, Artificial sequence; identical to SEQ ID NO: 2
  Oligonucleotides

SEQ ID NO: 72 - DNA, 21 bp, Artificial sequence
  Oligonucleotides
ttttttttttcggacgtcgtt

SEQ ID NO: 73 - DNA, 20 bp, Artificial sequence
  Oligonucleotides
tcggtcgatgttttcggtaa

SEQ ID NO: 74 - DNA, 110 bp, Artificial sequence; identical to SEQ ID NO: 5
  Olignucleotides

SEQ ID NO: 75 - DNA, 20 bp, Artificial sequence
  Oligonucleotides
ggagtggaggaaattgagat

SEQ ID NO: 76 - DNA, 22 bp, Artificial sequence
  Oligonucleotides
ccacacaacaaatactcaaaac

SEQ ID NO: 77 - DNA, 33 bp, Artificial sequence
  Oligonucleotides
tgggtgtttgtaatttttgttttgtgttaggtt

SEQ ID NO: 78 - DNA, 130 bp, Artificial sequence
  Oligonucleotides
ggagtggaggaaattgagatttattgaggttacgtagtttgtttaaggttaagtttgggtgtttgtaatttttgttttgtgttaggttgttttttaggtgttaggtgagttttgagtatttgttgtgtgg

(19) United States
(12) Patent Application Publication
(10) Pub. No.: US 2011/0027834 A1
(43) Pub. Date: Feb. 3, 2011

(54) Carry-Over Protection in Enzyme-Based Dna Amplification Systems Targeting Methylation Analysis

(76) Inventors: Reimo Tetzner, Berlin (DE); Dimo Dietrich, Berlin (DE)

Correspondence Address: Epigenomics AG, Kleine Praesidentenstr. 1, Berlin, 10178, DE

(21) Appl. No.: 12/308,149

(22) PCT Filed: Jun. 8, 2007
(86) PCT No.: PCT/EP2007/005088

§ 371 Date: Jul. 27, 2009

(30) Foreign Application Priority Data
Jun. 8, 2006 (EP) 06090102.2

(51) Int. Cl. C12P 19/34; C12N 9/00; C07H 21/04

(52) U.S. Cl. 435/91.5; 435/183; 536/24.33

(57) ABSTRACT

The invention refers to a method for providing a decontaminated template nucleic acid for enzymatic amplification reactions suitable for DNA methylation analysis. This method is characterized by the following steps: a) incubating a nucleic acid with a chemical reagent or an enzyme-containing solution, whereby the unmethylated cytosine bases are converted into uracil bases, b) mixing the template nucleic acid from step a) with the components required for an enzyme-mediated amplification reaction, including at least two oligonucleotides, whereby at least one of said oligonucleotides comprises i) at least one sequence part that hybridizes with a sequence of the template nucleic acid to be amplified, and ii) at least one sequence part that constitutes a recognition site for a DNA cleaving enzyme that cleaves DNA downstream of said recognition site and c) adding to this mixture a DNA cleaving enzyme, which specifically binds to the at least one sequence part that is a recognition site, and d) incubating the mixture, whereby nucleic acids containing said recognition site for a DNA cleaving enzyme are degraded.

The present invention relates to a method for providing a decontaminated template nucleic acid for enzyme-mediated amplification reactions suitable for DNA methylation analysis. The term “decontaminated” refers particularly to a template nucleic acid that is free of PCR-products stemming from previous amplification reactions (carry-over products).

Throughout this application, various publications are cited. The disclosure of these publications is hereby incorporated by reference in its entirety into this application to describe more fully the state of the art to which this invention pertains.

Gene regulation has been correlated with methylation of a gene or genome. Certain cell types consistently display specific methylation patterns, and this has been shown for a number of different cell types (Adorjan et al. (2002) Tumor class prediction and discovery by microarray-based DNA methylation analysis. Nucleic Acids Res 30(5) e21).

In higher order eukaryotes, DNA is methylated nearly exclusively at cytosine bases located 5′ to guanine in the CpG dinucleotide. This modification has important regulatory effects on gene expression, especially when involving CpG rich areas, known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosome of females.

The modification of cytosine in the form of methylation contains significant information. The identification of 5-methylcytosine within a DNA sequence is of importance in order to uncover its role in gene regulation. The position of a 5-methylcytosine cannot be identified by a normal sequencing reaction, since it behaves just as an unmethylated cytosine as per its hybridization preference.

Furthermore, in any standard amplification, such as a standard polymerase chain reaction (PCR), this relevant epigenetic information will be lost.

Several methods are known to solve this problem. Generally, genomic DNA is treated with a chemical or enzyme leading to a conversion of the cytosine bases, which consequently allows distinguishing between methylated and unmethylated cytosine bases. The most common methods are a) the use of methylation-sensitive restriction enzymes capable of differentiating between methylated and unmethylated DNA and b) the treatment with bisulfite. The use of methylation-sensitive restriction enzymes is limited due to the selectivity of the restriction enzyme towards a specific recognition sequence.

Therefore, the ‘bisulfite treatment’, allowing for the specific reaction of bisulfite with cytosine (which upon subsequent alkaline hydrolysis is converted to uracil, whereas 5-methylcytosine remains unmodified under these conditions (Shapiro et al. (1970) Nature 227: 1047)) is currently the most frequently used method for analyzing DNA for the presence of 5-methylcytosine. Uracil corresponds to thymine in its base pairing behavior, that is, it hybridizes to adenine. 5-methylcytosine does not change its chemical properties under this treatment and therefore still hybridizes with guanine. Consequently, the original DNA is converted in such a manner that 5-methylcytosine, which originally could not be distinguished from cytosine by its hybridization behavior, can now be detected as the only remaining cytosine using conventional molecular biological techniques, such as amplification and hybridization or sequencing. All of these techniques are based on base pairing, which can now be fully exploited. Comparing the sequences of the DNA with and without bisulfite treatment allows easy identification of those cytosines that have been methylated.

An overview of the further known methods of detecting 5-methylcytosine may be gathered from the following review article: Fraga F M, Esteller M, Biotechniques (2002) 33(3):632, 634, 636-649.

As the use of methylation-specific enzymes is dependent on the presence of restriction sites, most methods are based on a bisulfite treatment that is conducted before a detection or amplifying step (for review: DE 100 29 915 A1, page 2, lines 35-46 or the according translated U.S. application Ser. No. 10/311,661; see also WO 2004/067545). The term ‘bisulfite treatment’ is meant to comprise treatment with a bisulfite, a disulfite or a hydrogensulfite solution. As known to the expert skilled in the art and according to the invention, the term “bisulfite” is used interchangeably for “hydrogensulfite”.

Several laboratory protocols are known in the art, all of which comprise of the following steps: The genomic DNA is isolated, denatured, converted several hours by a concentrated bisulfite solution and finally desulfonated and desalted (e.g.: Frommer et al. (1992) A genomic sequencing protocol that yields a positive display of 5-methylcytosine residues in individual DNA strands. Proc Natl Acad Sci U.S.A.; 89(5): 1827-1831).

Subsequent to a bisulfite treatment, usually short, specific fragments of a known gene are amplified and either completely sequenced (Olek A, Walter J. (1997) The pre-implantation ontogeny of the H19 methylation imprint. Nat. Genet. 3: 275-6) or individual cytosine positions are detected by a primer extension reaction (Gonzalgo M L and Jones P A (1997) Rapid quantitation of methylation differences at specific sites using methylation-sensitive single nucleotide primer extension (Ms-SNuPE). Nucleic Acids Res 25: 2529-2531, WO 95/00669) or by enzymatic digestion (Xiong Z, Laird P W. (1997) COBRA: a sensitive and quantitative DNA methylation assay. Nucleic Acids Res 25: 2535-2534).

Another technique to detect hypermethylation is the so-called methylation specific PCR (MSP) (Herman J G, Graff J R, Myohanen S, Nelkin B D and Baylin S B. (1996), Methylation-specific PCR: a novel PCR assay for methylation status of CpG islands. Proc Natl Acad Sci USA. 93: 9821-6). The technique is based on the use of primer's that differentiate between a methylated and a non-methylated sequence if applied after bisulfite treatment of said DNA sequence. The primer either contains a guanine at the position corresponding to the cytosine in which case it will after bisulfite treatment only bind if the position was methylated. Or the primer contains an adenine at the corresponding cytosine position and therefore only binds to said DNA sequence after bisulfite treatment if the cytosine was unmethylated and has hence been altered by the bisulfite treatment so that it hybridizes to adenine. With the use of these primers, amplicons can be produced specifically depending on the methylation status of a certain cytosine and will as such indicate its methylation state.

Another technique is the detection of methylation via a labeled probe, such as used in the so called Taqman PCR, also known as MethyLight (U.S. Pat. No. 6,331,393 B1). With this technique it is feasible to determine the methylation state of single or of several positions directly during PCR, without having to analyze the PCR products in an additional step.

In addition, detection by hybridization has also been described (Olek et al., WO 99/28498).

The treatment with bisulfite (or similar chemical agents or enzymes) with the effect of altering the base pairing behavior of one type of cytosine specifically, either the methylated or the unmethylated, thereby introducing different hybridization properties, makes the treated DNA more applicable to the conventional methods of molecular biology, especially the polymerase-based amplification methods, such as PCR.

Base excision repair occurs in vivo to repair DNA base damage involving relatively minor disturbances in the helical DNA structure, such as deaminated, oxidized, alkylated or absent bases. Numerous DNA glycosylases are known in the art, and function in vivo during base excision repair to release damaged or modified bases by cleavage of the glycosidic bond that links such bases to the sugar phosphate backbone of DNA (Memisoglu, Samson (2000) Mutation Res. 451: 39-51). All DNA glycosylases cleave glycosidic bonds but differ in their base substrate specificity and in their reaction mechanisms.

One widely recognized application of such glycosylases is decontamination in PCR applications. In any such PCR amplification, 2 to the power of 30 (2³⁰) or more copies of a single template are generated. This very large amount of DNA produced helps in the subsequent analysis, like in DNA sequencing according to the Sanger method, but it can also become a problem when this amount of DNA is handled in an analytical laboratory. Even very small reaction volumes, when inadvertently not kept in a closed vial, can lead to contamination of the whole work environment with a huge number of DNA copies. These DNA copies may be templates for a subsequent amplification experiment performed, and the DNA analyzed subsequently may not be the actual sample DNA, but contaminating DNA from a previous experiment. This may also lead to false positive controls. That is, samples that should not contain any DNA, and therefore no amplification should be observed, still show DNA amplification.

In practice, this problem can be so persistent such that whole laboratories must move to a new location, because contamination of the work environment makes it impossible to carry out meaningful PCR based experiments. In a clinical laboratory, however, the concern is also that contaminating DNA may cause false results when performing molecular diagnostics. This would mean that contaminating DNA that stems from a previous patient is in fact analyzed, instead of the actual sample to be investigated.

Therefore, measures have been implemented to avoid contamination. This involves, for example, a PCR amplification and detection in one tube in a real time PCR experiment. In this case, it is not required that a PCR tube be opened. After use, the tube will be kept closed and discarded and therefore the danger of contamination leading to false results is greatly reduced.

However, molecular means are known in the art that reduce the risk of contamination. In a polymerase chain reaction, the enzyme uracil-DNA-glycosylase (UNG) can reduce the potential for false positive reactions due to amplicon carryover (see e.g. U.S. Pat. No. 5,035,996, or Thornton C G, Hartley J L, Rashtchian A (1992). Utilizing uracil DNA glycosylase to control carryover contamination in PCR: characterization of residual UDG activity following thermal cycling. Biotechniques. 13(2): 180-184). The principle of this contamination protection method is that in any amplification instead of dTTP, dUTP is provided and incorporated and the resulting amplicon can be distinguished from its template and any future sample DNA by uracil being present instead of thymine. Prior to any subsequent amplification, uracil DNA-glycosylase (UNG) is used to cleave these bases from any contaminating DNA, and therefore only the legitimate template remains intact and can be amplified.

This method is considered the standard method of choice in the art and is widely used in DNA based diagnostics. The following is a citation from a publication that summarizes the use of UNG (Longo M C, Berninger M S, Hartley J L (1990). Use of uracil DNA glycosylase to control carry-over contamination in polymerase chain reactions. Gene 93 (1): 125-128).

“Polymerase chain reactions (PCRs) synthesize abundant amplification products. Contamination of new PCRs with trace amounts of these products, called carry-over contamination, yields false positive results. Carry-over contamination from some previous PCR can be a significant problem, due both to the abundance of PCR products, and to the ideal structure of the contaminant material for re-amplification. We report that carry-over contamination can be controlled by the following two steps: (i) incorporating dUTP in all PCR products (by substituting dUTP for dTTP, or by incorporating uracil during synthesis of the oligodeoxyribonucleotide primers); and (ii) treating all subsequent fully preassembled starting reactions with uracil DNA glycosylase (UNG), followed by thermal inactivation of UNG. UNG cleaves the uracil base from the phosphodiester backbone of uracil-containing DNA, but has no effect on natural (i.e., thymine-containing) DNA. The resulting apyrimidinic sites block replication by DNA polymerases, and are very labile to acid/base hydrolysis. Because UNG does not react with dUTP, and is also inactivated by heat denaturation prior to the actual PCR, carry-over contamination of PCRs can be controlled effectively if the contaminants contain uracils in place of thymines.”

As the existence of uracil bases is an inherent feature of bisulfite converted DNA and the necessary property relied upon for detecting methylation differences, the method of choice for carry over protection based on uracil-DNA-glycosylase enzyme activity as described above cannot be applied. However, a number of powerful assays for diagnosis are based on PCR which is performed on bisulfite-converted DNA as a template. Therefore, for the routine performance of such assays in a laboratory, new methods for carry over prevention need to be developed. There is a great need in the art to provide solutions to the problem of how to achieve a reliable carry over protection when analyzing methylation of cytosine positions in DNA from patient samples.

The difficulty of solving the problem for decontamination of bisulfite converted templates is considered a general one, that can not be solved by adaptation of the standard UNG method, as any bisulfite converted DNA will contain uracil bases as well.

It is known from Ashkenas J et al. (2005) “Simple enzymatic means to neutralize DNA contamination in nucleic acid amplification.” BioTechniques 39: 69-73, to use restriction enzymes to neutralize carry-over contaminants that are present in PCR reactions. For that purpose, the amplification is performed using primers that contain a recognition site for a Type II restriction enzyme in the 5′ end of each PCR primer, permitting the resulting PCR product to be cleaved by the corresponding restriction enzyme. The truncated product is thus neutralized, in that it cannot be amplified using the original primer.

PCT/US2005/021525 describes carry-over for example with use of a particular restriction enzyme that cuts the amplificate at a restriction site between the primer sites. The limitation of this is that one cannot use any amplificate for analysis, but only those which bear such a particular restriction site.

A recognition sequence or recognition site as used herein is defined to refer to a DNA sequence (or subset thereof) that exhibits binding specificity for a DNA-binding protein motif of a protein.

It was, however, not obvious for a person skilled in the art to use this method for the invesligation of the methylation state of genetic material, mainly for two reasons: 1) The chemical and physio-chemical properties of native DNA and bisulfite-treated DNA are markedly different and therefore do not allow direct transfer of a method for native DNA to a method using bisulfite-treated DNA. 2) Whereas primers used on native DNA comprise in most cases four different nucleobases, primers used on bisulfite-treated DNA usually comprise only three bases, namely either adenine, cytosine and thymine, or guanine, adenine and thymine, respectively, due to conversion of cytosine bases into uracil bases, which behave like thymine bases with regard to their base pairing behavior. Therefore, the construction of primers that include enzyme recognition sites is more demanding.

It was therefore surprising that primers containing a recognition site for a Type II restriction enzyme can also be used in the analysis of the methylation state of genetic material and it was not obvious to apply this technology of using restriction enzymes to neutralize carry-over contaminants that are present in nucleic acid amplification reactions on bisulfate-treated DNA.

The problem underlying the present invention therefore was to provide a method for carry-over protection in polymerase-based DNA amplification systems targeting methylation analysis that could be performed in an easy and reliable manner without making use of the uracil-DNA-glycosylase enzyme.

Surprisingly, the inventors were able to solve this problem by inventing the present method. The central idea of the invention is to provide at least one oligonucleotide that comprises at least one sequence part that hybridizes with a sequence of the template nucleic acid to be amplified, and at least one sequence part that constitutes a recognition site for a DNA cleaving enzyme that cleaves DNA downstream of said recognition site, and to use this at least one oligonucleotide in an enzyme-based amplification reaction (e.g. a polymerase-based amplification) after incubating the template nucleic acid to be amplified with a chemical reagent (e.g. a bisulfite reagent containing solution) or an enzyme containing solution, whereby the unmethylated cytosine bases are converted into uracil bases. Prior to performing an enzyme-based amplification, a DNA cleaving enzyme is added, which specifically binds to the at least one sequence part that is a recognition site, and during an incubation period, nucleic acids containing said recognition site for a DNA cleaving enzyme are degraded. Therefore, the carry-over of DNA from previous experiments that were performed using the same oligonucleotide as a primer is prevented by cleaving such DNA molecules.

One embodiment of this invention therefore comprises a method which provides both a sufficient and reliable differentiation between methylated and unmethylated cytosines by using an enzyme-based DNA amplification assay, such as a common PCR-based assay. A further embodiment of this invention comprises a test kit which can be used for the realization of the method. Yet a further embodiment of this invention comprises the use of said method or of said test kit for diagnosis and/or prognosis of adverse events for patients or individuals. A still further embodiment of this invention comprises the use of at least one oligonucleotide as a primer for providing a decontaminated template nucleic acid for enzyme-based amplification reactions suitable for DNA methylation analysis.

In a further embodiment of the current invention, a method for the prevention of carry-over contamination amplifications mediated by at least one ligase (e.g. Ligase-Chain Reaction, LCR) or based on transcription (e.g. NASBA™ 3SR™, TMA™) by means of Type II restriction enzymes is provided. In these amplifications, the UNG method is not or only poorly applicable, since amplification takes place without the introduction of nucleotides (Ligase Chain Reaction) or via RNA intermediates. This embodiment of the invention is not restricted to methylation analysis, but also applicable for the analysis of (non-converted) genomic DNA. For both amplifications, the application of Type II restriction enzymes has not yet been described. The use of Type II restriction enzymes provides a surprisingly simple mean to prevent carry-over contaminations.

### DESCRIPTION OF THE INVENTION

Disclosed is a method for the specific amplification of template DNA in the presence of potentially contaminating amplification products from previous amplification experiments, suitable for DNA methylation analysis.

This method will typically be carried out by performing at least the following steps in the given order:
a) Firstly, a template DNA that is to be analyzed with respect to its methylation status, is incubated with a chemical reagent or an enzyme-containing solution, whereby the unmethylated cytosine bases are converted into uracil bases. Preferably, a bisulfate reagent is applied. The base-conversion will later be described with reference to FIG. 1. As a template DNA, isolated genomic DNA is usually used. It is usually already denatured and therefore present in a single stranded mode.
b) Secondly, the template nucleic acid from step a), in which all non-methylated cytosine bases have been converted to uracil bases, is mixed with the components required for an enzyme-mediated amplification reaction, including at least two oligonucleotides. At least one of said oligonucleotides comprises at least one sequence part that hybridizes with a sequence of the template nucleic acid to be amplified, and said at least one oligonucleotide further comprises at least one sequence part that constitutes a recognition site for a DNA cleaving enzyme that cleaves DNA downstream of said recognition site.
c) Thirdly, a DNA cleaving enzyme (restriction enzyme) that cleaves DNA down-stream of its recognition site is added to this mixture, which specifically binds to the at least one sequence part that is a recognition site.
d) Fourth, the mixture is incubated, whereby nucleic acids containing said recognition site for a DNA cleaving enzyme are degraded.

Subsequently, the following steps can be performed:
e) The mixture described above is incubated at an increased temperature, whereby the DNA cleaving activity is terminated, and
f) the template nucleic acid is amplified.

The method is useful for decontamination of nucleic acid samples, or rather for avoiding amplification of carry-over products, in particular in the context of DNA methylation analysis.

Preferably, the template nucleic acid is genomic DNA. It is, however, also possible to use DNA from other sources, such as synthesized DNA that does not stem from a natural source.

In the first step of the method according to the invention (step a)), the DNA is chemically or enzymatically treated in such a way that all of the unmethylated cytosine bases are converted to uracil or another base which is dissimilar to cytosine in terms of base pairing behavior, while the 5-methylcytosine bases remain unchanged.

This is preferably achieved by means of treatment with a bisulfite reagent. The term “bisulfite reagent” refers to a reagent comprising bisulfite, disulfite, hydrogen sulfite or combinations thereof, useful as disclosed herein to distinguish between methylated and unmethyllated CpG dinucleotide sequences. Methods of said treatment are known in the art (e.g. PCT/EP 2004/011715).

It is preferred that the bisulfite treatment is conducted in the presence of denaturing solvents, such as, but not limited to, n-alkylenglycol, particularly diethylene glycol dimethyl ether (DME), or in the presence of dioxane or dioxane derivatives. In a preferred embodiment, the denaturing solvents are used in concentrations between 1% and 35% (v/v). It is also preferred that the bisulfite reaction is carried out in the presence of scavengers such as, but not limited to, chromane derivatives, e.g., 6-hydroxy-2,5,7,8, -tetramethylchromane 2-carboxylic acid or trihydroxybenzoe acid and derivates thereof, e.g. Gallic acid (see: PCT/EP2004/011715). The bisulfite conversion is preferably carried out at a reaction temperature between 30° C. and 70° C., whereby the temperature is increased to over 85° C. for short periods of times during the reaction (see: PCT/EP2004/011715). The bisulfite-treated DNA is preferably purified prior to the quantification. This may be conducted by any means known in the art, such as, but not limited to, ultrafiltration, preferably carried out by means of Microcon™ columns (manufactured by Millipore™). The purification is carried out according to a modified manufacturer's protocol (see: PCT/EP2004/011715).

It is also possible to conduct the conversion enzymatically, e.g. by use of methylation-specific cytidine deaminases (German Patent DE 103 31 107; PCT/EP2004/007052).

In the second step of the invention, the template nucleic acid from step a), in which all non-methylated cytosine bases have been converted to uracil bases, is mixed with the components required for an enzymatic amplification reaction, including at least two oligonucleotides. At least one of said oligonucleotides comprises at least one sequence part that hybridizes with a sequence of the template nucleic acid to be amplified, and said oligonucleotides further comprises at least one sequence part that constitutes a recognition site for a DNA cleaving enzyme that cleaves DNA downstream of said recognition site.

The oligonucleotides will be chosen such that they amplify a fragment of interest. It is particularly preferred that these oligonucleotides are designed to amplify a nucleic acid fragment of a template nucleic acid sample by means of a polymerase reaction, in particular a polymerase chain reaction, as known in the art. The oligonucleotides are therefore designed to anneal to the template nucleic acids, to form a double strand, following the Watson-Crick base pairing rules, and the length of these oligonucleotides will be selected such that they anneal at approximately the same temperature.

To achieve best results with the method according to the present invention, the following preferred embodiments with respect to the at least one oligonucleotides should be realized.

To increase efficiency of DNA cleavage of contaminating nucleic acids, the at least one of said oligonucleotides comprises several, preferably two to four, recognition sites for a DNA cleaving enzyme, preferably for the same cleaving enzyme. The recognition sites may be separated by one or a plurality of nucleotides, however, preferably, they are consecutive (i.e. without intervening nucleotides). In a particular preferred embodiment, the nucleotides are aligned to form a tandem repeat, without any nucleotides between the two adjacent recognition sites.

To enable efficient annealing of the oligonucleotides to the template nucleic acid, the at least one sequence part that hybridizes with a sequence of the template nucleic acid to be amplified of the at least one oligonucleotide is complementary to the sequence of the template nucleic acid it binds to. Furthermore, the at least one sequence part that hybridizes with a sequence of the template nucleic acid to be amplified of the at least one of said oligonucleotides should have a length of between 15 to 25 nucleotides.

In order to amplify only one strand of the template nucleic acid, it is preferred that the at least one sequence part that hybridizes with a sequence of the template nucleic acid of the at least one of said oligonucleotides comprises at least three nucleotides that hybridize to nucleotides on the template DNA which have been converted from a cytosine base to a uracil base during step a) of the method according to the present invention as described above.

The at least one sequence part that hybridizes with a sequence of the template nucleic acid, and the at least one sequence part that is a recognition site for a DNA cleaving enzyme can be separate from each other, that is to say, the sequences do not overlap. In that case, the at least one sequence part that is a recognition site should be located on the 5′-side of the oligonucleotide, and the at least one sequence part that hybridizes with a sequence of the template nucleic acid should be located on the 3′-side of the oligonucleotide. Thereby, it is ensured that the DNA cleaving enzyme cleaves contaminating DNA molecules within the sequence that the oligonucleotide anneals to or, which is preferred, within a sequence of the template, which is located downstream from the 3′ end of the sequence that the oligonucleotide anneals to. This way, the degradation of oligonucleotides is prevented, in particular when DNA cleaving enzymes are used that cleave single stranded DNA molecules.

It is also possible that both sequence parts overlap at least partially or are even identical. It may be, however, difficult to devise an oligonucleotide in which both sequence parts overlap or are identical, since an oligonucleotide that is complementary to the target nucleic acid after bisulfite treatment, can only contain three instead of four bases, namely adenine, cytosine and thymine, or guanine, adenine and thymine. Therefore, the number of possible recognition sites for possible DNA cleaving enzyme is limited to enzymes that recognize a recognition site consisting of only a set of the three different bases named above.

As known in the art, the at least one oligonucleotide may also comprise at least one modification, such as a fluorophore (e.g. FAM, HEX, ROX, Tamra), a quencher (e.g. BHQ1, BHQ1, Dabcyl), an inosine base, a Universal-Base, a dSpacer, a minor groove binder, a 5′- or 3′-phosphate-, amino- or didesoxy-nucleotide, a RNA nucleotide, or a LNA nucleotide.

The DNA cleaving enzyme, which is added in step c) as described above, specifically binds to the at least one sequence part that is a recognition site. It cleaves (single stranded and/or double stranded) DNA downstream of its recognition site, thereby removing the binding sequence to which the at least one oligonucleotide binds. Ideally, DNA cleavage takes place within a sequence of the nucleic acid that is located 3′ to the sequence part that hybridizes with the at least one oligonucleotide.

Therefore, not the target nucleic acid to be amplified will at this step be recognized and degraded by the enzyme, but only nucleic acids that were generated in preceding amplifications using primers with said DNA cleaving enzyme recognition site.

It is preferred that the DNA cleaving enzyme cleaves the DNA at least 10 nucleotides downstream from its recognition site. It is particularly preferred that the DNA cleaving enzyme cleaves the DNA at least 15 nucleotides, most preferably at least 20 nucleotides downstream from its recognition site.

This ensures that cleavage occurs outside of the primer sequence or at least that the part of the primer sequence that remains on the 3′ site of the cleavage site is so short that annealing of the oligonucleotides used does not occur and thus, no amplification product is generated from such templates.

The DNA cleaving enzyme that is added to the mixture in step c) binds specifically to the at least one sequence part that is a recognition site and cleaves the DNA downstream of this recognition site. This way all template DNA molecules that have been carried over as contaminants from previous experiments and contain this recognition site (since these previous experiments were also performed using oligonucleotides as primers that contained at least one recognition site for this DNA cleaving enzyme) are being cleaved.

Restriction enzyme systems have been subdivided into three categories: Type I, Type II and Type III (Yuan, R (1981) Annu Rev Biochem 50: 285; Smith HO (1979) Science 205: 455). The major differences are that Type II enzymes contain separate restriction and methylation systems, while Type I and Type III enzymes carry both restriction and methylation properties in one enzyme, consisting of two or three heterologous subunits. Type II restriction enzymes have a short, often palindromic, recognition site. A subset of Type II, the Type IIs restriction enzymes also have a short recognition sequence but it is rarely palindromic. In this case, the restriction enzyme will always cut downstream of the recognition sequence instead of within it. Therefore, DNA restriction enzymes Type IIs are preferred in the present invention.

It is particularly preferred that the DNA cleaving enzyme cleaves single stranded DNA, such as Mbo II. In order to avoid the breakdown of oligonucleotides that serve as primers, it is of importance to choose the single stranded DNA cleaving enzyme and the sequence hybridizing with the target DNA such that cleavage of the nucleic acid occurs so far down-stream that unhybridized primers are not degraded.

Preferred DNA cleaving enzymes are listed in table 3.

Preferably, an amount of units of the DNA cleaving enzyme is added, that is required to degrade essentially all potential contaminants. This ensures complete degradation of contamination nucleic acids stemming from amplification reactions performed previously using the same oligonucleotides.

After enzymatic degradation, the DNA cleaving enzyme is subsequently inactivated, preferably by heat, so that it is not capable of substantially cleaving any product of the subsequent amplification step.

In a preferred embodiment of the invention, the enzyme-based amplification reaction is started by heat-activation, that is, by a brief incubation at increased temperature which activates the enzymatic activity. For this purpose, a heat stable enzyme is preferred. This heat-activation can at the same time deactivate the DNA cleaving enzyme.

The template nucleic acid may now be amplified enzymatically in the next step, while any cleaved contaminating DNA is essentially not amplified. In case the enzyme-based amplification reaction is a polymerase-based amplification reaction, in particular a polymerase chain reaction (PCR), it is preferred that the enzyme is a heat stable polymerase. However, it is also possible to apply other enzymatic amplification reactions known to the person skilled in the art, including, but not limited to, ligase-mediated amplifications (e.g. Ligase-Chain Reaction) or amplifications based on transcription (e.g. NASBA™ 3 SR™, TMA™).

The amplified products may be analyzed and the methylation status in the genomic DNA may be deduced from the presence of an amplified product and/or from the analysis of the sequence within the amplified product.

The generated DNA fragments will then be analyzed, concerning their presence, the amount, or their sequence properties or a combination thereof.

It is preferred that upon activation of the polymerase enzyme, a polymerase based amplification reaction or an amplification based assay is performed. It is further preferred that this assay is performed in the real time formate.

The sample DNA may be obtained from serum or other body fluids of an individual. The sample DNA may, for example, be obtained from cell lines, tissue embedded in paraffin, such as tissue from eyes, intestine, kidneys, brain, heart, prostate, lungs, breast or liver, histological slides, body fluids and all possible combinations thereof.

The term body fluids is meant to comprise fluids such as whole blood, blood plasma, blood serum, urine, sputum, ejaculate, semen, tears, sweat, saliva, lymph fluid, bronchial lavage, pleural effusion, peritoneal fluid, meningal fluid, amniotic fluid, glandular fluid, fine needle aspirates, nipple aspirate fluid, spinal fluid, conjunctival fluid, vaginal fluid, duodenal juice, pancreatic juice, bile, stool and cerebrospinal fluid. It is especially preferred that said body fluids are whole blood, blood plasma, blood serum, urine, stool, ejaculate, bronchial lavage, vaginal fluid and nipple aspirate fluid.

The following methylation detection assays may all be performed subsequently to the steps of the method according to the invention:

Methylation Assay Procedures. Various methylation assay procedures are known in the art, and can be used in conjunction with the present invention. These assays allow for determination of the methylation state of one or a plurality of CpG dinucleotides (e.g., CpG islands) within a DNA sequence. Such assays involve, among other techniques, DNA sequencing of bisulfite-treated DNA, and a number of PCR based methylation assays, some of them—known as COBRA, MS-SNuPE, MSP, nested MSP, HeavyMethyl and MethyLight—are described in more detail now.

BISULFITE SEQUENCING. DNA methylation patterns and 5-methylcytosine distribution can be analyzed by sequencing analysis of a previously amplified fragment of the bisulfite treated genomic DNA, as described by Frommer et al. (Frommer et al. Proc. Natl. Acad. Sci. USA 89: 1827-1831, 1992). As the bisulfite treated DNA is amplified before sequencing, the amplification procedure according to the invention may be used in combination with this detection method.

COBRA. COBRA analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992) or as described by Olek et al. (Olek A, Oswald J, Walter J. (1996) Nucleic Acids Res. 24: 5064-6). PCR amplification of the bisulfite converted DNA is then performed using methylation unspecific primers followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Additionally, restriction enzyme digestion of PCR products amplified from bisulfite-converted DNA is also used, in the method described by Sadri & Hornsby (Nucl. Acids Res. 24:5058-5059, 1996)

The bisulfite conversion and amplification procedure according to the invention may be used in combination with this detection method.

Ms-SNuPE (Methylation-sensitive Single Nucleotide Primer Extension). The Ms-SNuPE technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites.

Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

The bisulfite conversion and amplification procedure according to the invention may be used in combination with this detection method.

MSP. MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus unmethylated DNA.

MSP primer pairs contain at least one primer, which hybridizes to a bisulfite treated CpG dinucleotide. Therefore, the sequence of said primers comprises at least one CpG dinucleotide. MSP primers specific for non-methylated DNA contain a “T′ at the 3′ position of the C position in the CpG. Preferably, therefore, the base sequence of said primers is required to comprise a sequence having a length of at least 9 nucleotides which hybridizes to the bisulfite converted nucleic acid sequence, wherein the base sequence of said oligomers comprises at least one CpG dinucleotide. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes.

The bisulfite conversion and amplification procedure according to the invention may be used in combination with this detection method.

NESTED MSP (Belinsky and Palmisano in US application 20040038245). Considering the apparent conflict of requiring high specificity of the MSP primer to sufficiently differentiate between CG and TG positions but allowing for a mismatch in order to create a unique restriction site it is preferred to use an amended version of MSP, known as nested MSP, as described in WO 02/18649 and US patent application 20040038245 by Belinsky and Palmisano. This method to detect the presence of gene-specific promoter methylation, comprises the steps of: expanding the number of copies of the genetic region of interest by using a polymerase chain reaction to amplify a portion of said region where the promoter methylation resides, thereby generating an amplification product; and using an aliquot of the amplification product generated by the first polymerase chain reaction in a second, methylation-specific, polymerase chain reaction to detect the presence of methylation. In other words a non methylation specific PCR is performed prior to the methylation specific PCR. The bisulfite conversion and amplification procedure according to the invention may be used in combination with this detection method.

HEAVYMETHYL. (WO 02/072880; Cottrell S E et al. Nucleic Acids Res. 2004 Jan. 13; 32(1):e10) A further preferred embodiment of the method comprises the use of blocker oligonucleotides. In the HeavyMethyl assay blocking probe oligonucleotides are hybridized to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulfite treated nucleic acid in a methylation status specific manner. For example, for detection of methyllated nucleic acids within a population of unmethylated nucleic acids, suppression of the amplification of nucleic acids which are unmethylated at the position in question would be carried out by the use of blocking probes comprising a ‘CpA’ or ‘TpA’ at the position in question, as opposed to a ‘CpG’ if the suppression of amplification of methylated nucleic acids is desired.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivatized at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-terminii thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

Preferably, therefore, the base sequence of said blocking oligonucleotide is required to comprise a sequence having a length of at least 9 nucleotides which hybridizes to the chemically treated nucleic acid sequence, wherein the base sequence of said oligonucleotides comprises at least one CpG, TpG or CpA dinucleotide.

The bisulfite conversion and amplification procedure according to the invention may be used in combination with this detection method.

Preferably, real-time PCR assays are performed specified by the use of such primers according to the invention. Real-time PCR assays can be performed with methylation specific primers (MSP-real time) as methylation-specific PCR (“MSP”; as described above), or with non-methylation specific primers in presence of methylation specific blockers (HM real-time) (“HEAVYMETHYL”, as described above). Real-time PCR may be performed with any suitable detectably labeled probes. For details, see below.

Both of these methods (MSP or HM) can be combined with the detection method known as MethyLight™ (a fluorescence-based real-time PCR technique) (Eads et al., Cancer Res.

59:2302-2306, 1999), which generally increases the specificity of the signal generated in such an assay. Whenever the real-time probe used is methylation specific in itself, the technology shall be referred to as MethyLight™, a widely used method.

Another assay makes use of the methylation specific probe, the so called “QM” (quantitative methylation) assay. A methylation unspecific, therefore unbiased real-time PCR amplification is performed which is accompanied by the use of two methylation specific probes (MethyLight™) one for the methylated and a second for the unmethylated amplificate. That way two signals are generated which can be used to a) determine the ratio of methylated (CG) to unmethylated (TG) nucleic acids, and at the same time b) the absolute amount of methylated nucleic acids can be determined, when calibrating the assay with a known amount of control DNA.

MethyLight™. The MethyLight™ assay is a high-throughput quantitative methylation assay that utilizes fluorescence-based real-time PCR (TaqMan™) technology that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts un-methylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both.

The MethyLight™ assay may be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methyllation sites.

The MethyLight™ process can by used with a “TaqMan®” probe in the amplification process. For example, double-stranded genomic DNA is treated with sodium bisulfite and subjected to one of two sets of PCR reactions using TaqMan® probes; e.g., with either biased primers and TaqMan® probe, or unbiased primers and TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Variations on the TaqMan™ detection methodology that are also suitable for use with the described invention include the use of dual-probe technology (LightCycler™) or fluorescent amplification primers (Sunrise™ technology). Both these techniques may be adapted in a manner suitable for use with bisulfite treated DNA, and moreover for methylation analysis within CpG dinucleotides.

Typical reagents (e.g., as might be found in a typical MethyLight™-based kit) for MethyLight™ analysis may include, but are not limited to: PCR primers for specific bisulfite sequences, i.e. bisulfite converted genetic regions (or bisulfite converted DNA or bisulfite converted CpG islands); probes (e.g. TaqMan® or LightCycler™) specific for said amplified bisulfite converted sequences; optimized PCR buffers and deoxynucleotides; and a polymerase, such as Taq polymerase. A further real time technology for methylation analysis is Scorpio™ (WO 2005/024056).

The bisulfite conversion and amplification procedure according to the invention may be used in combination with this detection method.

The fragments obtained by means of the amplification can carry a directly or indirectly detectable label. Preferred are labels in the form of fluorescence labels, radionuclides, or detachable molecule fragments having a typical mass, which can be detected in a mass spectrometer. Where said labels are mass labels, it is preferred that the labeled amplificates have a single positive or negative net charge, allowing for better detectability in the mass spectrometer. The detection may be carried out and visualized by means of, e.g., matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas & Hillenkamp, (1988) Anal Chem 60: 2299-2301). An analyte is embedded in a light-absorbing matrix. The matrix is evaporated by a short laser pulse thus transporting the analyte molecule into the vapor phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut & Beck, Current Innovations and Future Trends, 1: 147-157, 1995). The sensitivity with respect to nucleic acid analysis is approximately 100-times less than for peptides, and decreases disproportionally with increasing fragment size. Moreover, for nucleic acids having a multiply negatively charged backbone, the ionization process via the matrix is considerably less efficient. In MALDI-TOF spectrometry, the selection of the matrix plays an eminently important role. For desorption of peptides, several very efficient matrixes have been found which produce a very fine crystallization. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge-tag to this modified DNA results in an increase in MALDI-TOF sensitivity to the same level as that found for peptides.

The amplificates may also be further detected and/or analyzed by means of oligonucleotides constituting all or part of an “array” or “DNA chip” (i.e., an arrangement of different oligonucleotides and/or PNA-oligomers bound to a solid phase). Such an array of different oligonucleotide- and/or PNA-oligomer sequences can be characterized, for example, in that it is arranged on the solid phase in the form of a rectangular or hexagonal lattice. The solid-phase surface may be composed of silicon, glass, polystyrene, aluminum, steel, iron, copper, nickel, silver, or gold. Nitrocellulose as well as plastics such as nylon, which can exist in the form of pellets or also as resin matrices, may also be used. An overview of the Prior Art in oligomer array manufacturing can be gathered from a special edition of Nature Genetics (Nature Genetics Supplement, Volume 21, January 1999, and from the literature cited therein). Fluorescently labeled probes are often used for the scanning of immobilized DNA arrays. The simple attachment of Cy3 and Cy5 dyes to the 5′-OH of the specific probe is particularly suitable for fluorescence labels. The detection of the fluorescence of the hybridized probes may be carried out, for example, via a confocal microscope. Cy3 and Cy5 dyes, besides many others, are commercially available.

The base conversion and amplification procedure according to the invention may be used in combination with this detection method.

A particular preferred embodiment of the invention is a method for providing a decontaminated nucleic acid for hybridization on a DNA-Array, preferably an Oligonucleotide-Array, suitable for DNA methylation analysis.

A further embodiment of the invention provides a method for providing a decontaminated template nucleic acid for enzymatic amplification reactions. According to this embodiment, a template nucleic acid is mixed with the components required for an amplification, mediated by at least one ligase or based on transcription, including at least two oligonucleotides, whereby at least one of said oligonucleotides comprises at least one sequence part that hybridizes with a sequence of the template nucleic acid to be amplified, and also at least one sequence part that constitutes a recognition site for a DNA cleaving enzyme that cleaves DNA downstream of said recognition site. In a further step, a DNA cleaving enzyme is added to this mixture, which specifically binds to the at least one sequence part that is a recognition site, and the mixture is incubated, whereby nucleic acids containing said recognition site for a DNA cleaving enzyme are degraded.

In a preferred embodiment of this method, the following subsequent steps are conducted: incubating the mixture at an increased temperature, whereby the enzymatic DNA cleaving activity is terminated, and amplifying the template nucleic acid.

In a further embodiment of the present invention, a test kit for the realization of the method comprises a chemical reagent or an enzyme that converts unmethylated cytosine bases into uracil bases, particularly a bisulfite-containing component, for example a reagent or solution containing bisulfite, and a component containing an enzymatic activity. This enzymatic activity specifically binds to a sequence recognition site and cleaves DNA down-stream from this recognition site. A further component of the test kit is at least one oligonucleotide which comprises i) at least one sequence part that hybridizes with a sequence of the template nucleic acid to be amplified, and ii) at least one sequence part that constitutes a recognition site for a DNA cleaving enzyme that cleaves DNA downstream of said recognition site. This at least one oligonucleotide enables the specific cleavage of contaminating nucleic acids that stem from previous amplification experiments using similar oligonucleotides to be specifically cleaved by the enzymatic activity in the form of a DNA cleaving enzyme.

The test kit may further comprise one or more of the additional components, such as:
one or more denaturing reagent and/or solution, for example: dioxane or diethylene glycol dimethylether (DME) or any substance, which is suitable as described in WO 05/038051,
one or more scavenger, for example 6-hydroxy-2,5,7,8-tetramethylchromane 2-carboxylic acid or other scavengers as described in WO 01/98528 or WO 05/038051,
at least one additional primer, which is suitable for the amplification of one or more

DNA amplificates. The primer or primers can be modified, for example with a quencher and/or a label for detection as well known by a person skilled in the art like the dye FAM or the quencher BHQ black hole or dabcyl,
one or more probes, which can be any probe, which can be used to specifically record the amplification of one or more amplificates for example in a real-time-assay, amongst others the probe or probes can be modified, for example with a quencher and/or a label for detection as well known by a person skilled in the art like the dye FAM or the quencher BHQ black hole or dabcyl,
one or more blockers, which are nucleic acids and can be used to block the binding of a specific primer or the replication by DNA polymerase, amongst others the blocker or blockers can be modified, for example with a quencher and/or a label for detection as well known by a person skilled in the art like the dye FAM or the quencher BHQ black hole or dabcyl,
one or more reaction buffers, which are suitable for a bisulfite treatment and/or a PCR reaction,
nucleotides, which can be dATP, dCTP, dTTG, dUTP and dGTP or any derivative of these nucleotides,
MgCl₂ as a substance or in solution and/or any other magnesium salt, which can be used to carry out a DNA polymerase replication,
DNA polymerase, for example Taq polymerase or any other polymerase with or without proof-reading activity,
dye or quencher, which can be used for the detection of the amplificates as known in the art, for example an intercalating dye like SYBR Green or a dye for linkage to a primer or probe or blocker like the dye FAM or the quencher BHQ black hole or dabcyl, and/or
any reagent, solution, device and/or instruction which is useful for realization of a method according to the invention.

The methods and test kit disclosed here are preferably used for the diagnosis and/or prognosis of adverse events for patients or individuals, whereby diagnosis means diagnose of an adverse event, a predisposition for an adverse event and/or a progression of an adverse event. Regarding such a use of the method according to the invention, the method can be understood as a part of the methylation analysis on the results of which the diagnosis and/or prognosis of adverse events is based upon.

The adverse events mentioned above belong to at least one of the following categories: undesired drug interactions; cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction or damage; malfunction, damage or disease of the gastrointestinal tract; malfunction, damage or disease of the respiratory system; lesion, inflammation, infection, immunity and/or convalescence; malfunction, damage or disease of the body as an abnormality in the development process; malfunction, damage or disease of the skin, of the muscles, of the connective tissue or of the bones; endocrine and metabolic malfunction, damage or disease; headaches or sexual malfunction.

Examples of such adverse events with relation to methylation of genomic DNA is known to a person of skill in the art.

The methods and test kits also serve for distinguishing cell types and tissues or for investigating cell differentiation. They also serve for analyzing the response of a patient to a drug treatment.

The methods and test kit of the invention can also be used to characterize the DNA methyllation status in that positions are methylated or non-methylated compared to normal conditions if a single defined disease exists. In a particular preferred manner they can serve for identifying an indication-specific target, wherein a template nucleic acid is treated according to the method of the present invention, and wherein an indication-specific target is defined as differences in the DNA methylation status of a DNA derived from a diseased tissue in comparison to a DNA derived from a healthy tissue. These tissue samples can originate from diseased or healthy patients or from diseased or healthy adjacent tissue of the same patient.

The indication specific target can be a protein, peptide or enzyme, and in particular a per se known modulator of the coded protein, peptide or enzyme is assigned with the specific indication of the diseased tissue. This modulator can serve for preparing a pharmaceutical composition with a specific indication, in particular a specific cancer indication.

In a preferred embodiment of the present invention, an enzyme listed in table 3 is used for the generation of contamination free nucleic acids for methylation analysis.

In a further embodiment of the present invention, at least one oligonucleotide is used for providing a decontaminated template nucleic acid for polymerase-based amplification reactions suitable for DNA methylation analysis, in that said at least one oligonucleotide comprises i) at least one sequence part that hybridizes with a sequence of the template nucleic acid to be amplified, and ii) at least one sequence part that constitutes a recognition site for a DNA cleaving enzyme that cleaves DNA downstream of said recognition site.

Finally, another embodiment of the invention is an oligonucleotide, particularly a primer oligonucleotide, which comprises i) at least one sequence part that hybridizes with a sequence of the template nucleic acid to be amplified, and ii) at least one sequence part that constitutes a recognition site for a DNA cleaving enzyme that cleaves DNA downstream of said recognition site, whereby the sequence part that hybridizes with the sequence to be amplified is bisulfite-specific (or specific to any other conversion reagent or enzyme), i.e. that this part of the sequence only contains the three nucleotides (C, A and T; or G, A and T).

### DESCRIPTION OF THE DRAWINGS

FIG. 1:

FIG. 1 describes the complete conversion of unmethylated cytosine to uracil, also referred to as bisulfite conversion, which is known in the art. The first step of this reaction takes place when unmethylated cytosine bases are contacted with hydrogensulfite at a pH around 5, and sulfonated at position C6.

The second step is the deamination that takes place rather spontaneously in aqueous solution. Thereby, cytosine sulfonate is converted into uracil sulfonate. The third step is the desulfonation step, which takes place in alkaline conditions, resulting in uracil.

FIG. 2:

FIG. 2 is a plot of real-time amplification of methylated DNA of the TMEFF2 gene from bisulfite converted DNA, according to the state of the art, without incubation with Gsu I restriction enzyme. The Y-axis shows the fluorescence signal measured in channel F2 normalized against channel F1 (channel 640 nm/530 nm) at each cycle (X-axis). The primers used contained a generic Gsu I restriction site (Seq-ID 1 and Seq-ID 2). The signals were obtained in the LightCycler instrument using specific hybridization probes generating a FRET signal (Seq-ID 3 and Sq-ID 4). Signals were obtained using the following templates, A: 1 ng methylated bisulfite converted DNA (solid line), B: 10⁴ copies of PCR product (triangles), C: 10³ copies of PCR product (rectangles). The control reaction without template showed no amplification (crosses).

FIG. 3:

FIG. 3 is a plot of real time amplification of methylated DNA of the TMEFF2 gene from bisulfite-converted DNA according to the method of the present invention, that is with prior treatment of the reaction mix using Gsu I restriction enzyme. The Y-axis shows the fluorescence signal measured in channel F2 normalized against channel F1 (channel 640 nm/530 nm) at each cycle (X-axis). The primers used contained a generic Gsu I restriction site (Seq-ID 1 and Seq-ID 2). The signals were obtained in the LightCycler instrument using specific hybridization probes generating a FRET signal (Seq-ID 3 and Sq-ID 4). Signals were obtained using the following template, A: 1 ng methylated bisulfite converted DNA (solid line), B: 10⁴ copies of PCR product (triangles). No amplification was observed using 10³ copies of PCR product (rectangles) and without template (crosses). Although the re-amplification of 10⁴ copies of PCR product took place, the obtained signals are significantly low and detected with a delay of 14 cycles compared to the reaction without Gsu I restriction enzyme (FIG. 1). In contrast to PCR products, the methylated bisulfite-converted DNA was amplified with the same efficiency compared to the reaction without Gsu I restriction enzyme.

FIG. 4:

FIG. 4 shows representative amplification curves of the reference PCR on 4 samples (18NT, 18TU, 24NT, 24TU). Amplification curves from colorectal cancer tissue are labeled with black cycles (18TU, 24TU), whereas DNA from normal tissue is shown in grey diamonds (18NT, 24NT). The no-template control is shown as dashed line.

FIG. 5:

FIG. 5 shows representative amplification curves of the TMEFF2 HeavyMethyl PCR on the same 4 samples (18NT, 18TU, 24NT, 24TU) shown in FIG. 4. Amplification curves from colorectal cancer tissue are labeled with black cycles (18TU, 24TU), whereas DNA from normal tissue are shown in grey diamonds (18NT, 24NT). No template control is shown as dashed line.

FIG. 6:

FIG. 6 shows the correlation of the methylation values (PMR) of the TMEFF2 gene in colorectal cancers and normal tissue obtained by two different methods. The PMR values were calculated from the proportion of the methylated DNA of the TMEFF2 gene according to the TMEFF2 HeavyMethyl assay against the total DNA. The values plotted on the X-axis are the results from samples added (“spiked”) with 3,000 copies of TMEFF2 PCR product, whereas the reaction mix contained 1 unit Gsu I endonuclease. The values plotted on the Y-axis were calculated from reactions without Gsu I, in which the samples were not contaminated with PCR products.

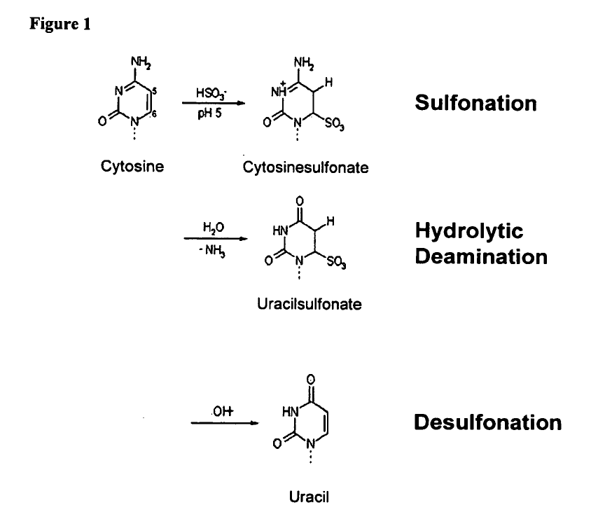

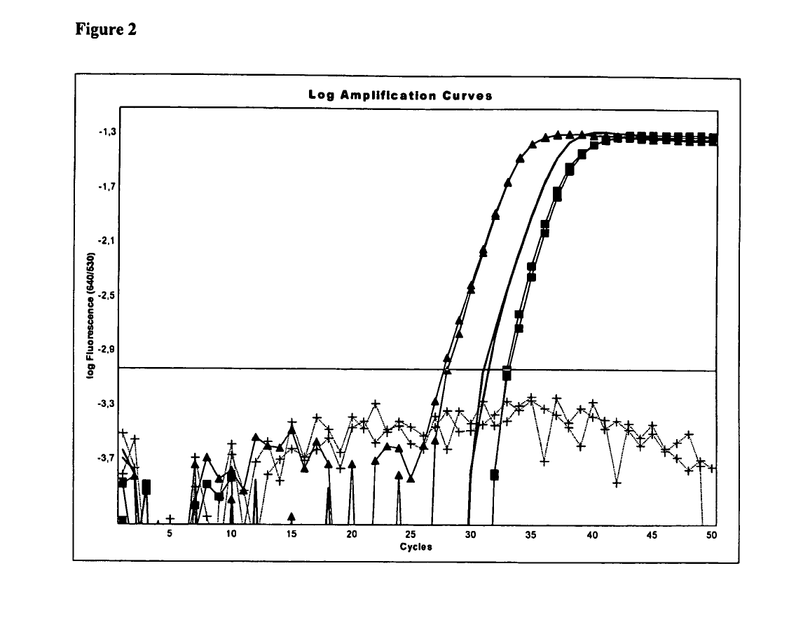

### EXAMPLES

### Example 1

### Detection of Methylated DNA of the TMEFF2 Gene Using Primers Comprising a Generic Gsu I-Recognition Site

The method according to the present invention allows the cleavage of contaminating PCR products stemming from previous amplification reactions, which would lead to false positive results of the analysis. The recognition of the contaminating PCR products is based on the introduction of a Gsu I recognition site in a tandem formation by introducing a generic 5′-domain into gene-specific primers.

The carry-over prevention procedure is realized by using such primers in every PCR, combined with the incubation of the PCR reaction mix with Gsu I prior to thermo-cycling. The closed reaction vessel contains all PCR components including the template DNA and does not to be reopened after the sterilization. In the following example it was shown that the method according to the present invention allows the use the Gsu I restriction endonuclease for carry-over prevention in polymerase-mediated reactions amplifying bisulfite-converted DNA without loss of sensitivity. To achieve this, the following steps were carried out:

1 μg GpGenome™ Universal Methylated DNA (Chemicon International, Temecula USA) was applied to a cytosine conversion procedure, where all unmethylated cytosines get converted to uracil, whereas all 5′-methylated cytosines remain unchanged. For this purpose, a bisulfite treatment was performed according to a method, which is state of the art (see for example WO 2005038051). Subsequently, the DNA was stored at 4° C. for several hours and then used as template in a PCR reaction.

Oligonucleotides for the amplification of a 110 by fragment of the promotor region of the TMEFF2 gene (GeneBank accession umber AF242221 nucleotide 1104 to 1214, Seq-ID 5) were designed by analyzing the theoretical DNA sequence after bisulfite conversion (Clark et al. (1994), Nucleic Acids Res. 22(15): 2990-2997.). Additionally, fluorescence-labeled probes were designed for the detection of the amplicon in a real-time PCR system, based on the FRET mechanism (Seq-ID 3 and Seq-ID 4). The real-time PCR was realized in the LightCycler instrument (Roche Diagnostics) and tested for specificity and efficiency, according to Cottrell et al. (2004), Nucleic Acids Res. 32(1): e10.

The sequences of the primer oligonucleotides were afterwards modified for the application of the invented Gsu I carry-over prevention system. For this purpose, the recognition site of the Gsu I endonuclease, which is 5′-CTGGAG, was added to the 5′-end in a tandem formation, which is 5′-CTGGAGCTGGAG. These modified primers were used for the TMEFF2 real-time assay, which was again tested for specificity and efficiency.

The carry-over prevention system was then employed on a TMEFF2 real-time PCR reaction by adding 1 unit Gsu I to the reaction mix combined with a pre-incubation of the closed reaction vessel for 30 min at 30° C. Two different reaction mixes were prepared containing either 1 unit Gsu I or not. Three different template DNAs were added to the mixtures in replicates: a) 1 ng bisulfite treated universal methylated DNA, b) 10³ copies and c) 10⁴ copies of the TMEFF2 amplicon, generated by use of the same primers (Seq-ID 1 and Seq-ID2) containing the Gsu I restriction site in a tandem formation. The real-time PCR was performed in the LightCycler instrument and the crossing points (Cp) were determined by the automated LightCycler software using the “second derivate methode”.

The reaction mix contained either 1 unit Gsu I, or no Gsu I was added. PCR reactions were performed in the LightCycler in 20 μl reaction volume and contained:

10 μl of template DNA or PCR products

2 μl of FastStart LightCycler Mix for Hybridization probes (Roche Diagnostics)

3.5 mM MgCl₂ (Roche Diagnostics)

0.30 μM forward primer (Seq-ID1, TIB-MolBiol)

0.30 μM reverse primer (Seq-ID2, TIB-MolBiol)

0.15 μM Probel (Seq-ID3, TIB-MolBiol)

0.15 μM Probe2 (Seq-ID4, TIB-MolBiol)

Optionally, 1 unit Gsu I restriction endonuclease (Fermentas, #ER0462)

The temperature time profile was programmed as follows:

| | |
|---|---|
| Preincubation (Gsu I active): | 30 min at 30° C. |
| Activation of polymerase | 10 min at 95° C. |
| (Gsu I inactivation): | |
| 50 temperature cycles: | 10 sec at 95° C. |
| | 30 sec at 56° C. |
| | 10 sec at 72° C. |

Finally, the reaction was cooled down to 40° C. The signals were obtained in each cycle during the annealing step at 56° C. by measuring the fluorescence signal at channel 640 nm normalized against the channel 530 nm. The oligonucleotides used are collected in table 1.

The results of the experiment are summarized in table 2. The re-amplification of 10³ copies of PCR product containing the Gsu I restriction site resulted in a Cp of 32.8 when Gsu I was present in the reaction mix, whereas no signal was detected in the reaction mixtures with Gsu I. The reaction containing 10⁴ copies of PCR product were detected at cycle 27.4 in the reaction without Gsu I and at cycle 41.2 in the reaction with Gsu I.

This demonstrates the efficient recognition and cleavage of spiked PCR products by the endonuclease. In contrast to this, bisulfite-treated sample DNA was amplified in both cases, without and with Gsu I restriction endonuclease with almost the same efficiency. Methylated bisulfite-treated DNA was detected at a Cp 30.4 and at Cp 30.8 with or without Gsu I, respectively. All Cp values presented are means from two experiments.

The re-amplification of the PCR products was significantly inhibited by the incubation of the master mix with Gsu I. No amplification could be observed from reactions with 1.000 copies of PCR product. Although 10.000 copies PCR products were detected, the signal to noise ratio of these samples remained very low and the measured Cp values were higher then 40 cycles, which represent a negative read-out in a standard real-time PCR. On the other hand, in this example it could be shown that the bisulfite-treated sample DNA was not affected by the pre-incubation with Gsu I, since it was working as a template in the amplification reaction in the same manner as observed without the addition of Gsu I.

TABLE 1
Sequences of oligonucleotides used in example 1

| SeqID | Name | Sequence |
|---|---|---|
| Seq-ID 1 | Gsu-TMEFF2 | 5′-CTGGAGCTGGAGAAaAaAAAaaCTCCTCTaCATAC |
| Seq-ID 2 | Gsu-TMEFF2 | 5′-CTGGAGCTGGAGGTtAtTGttTGGGttAAtAAATG |
| Seq-ID 3 | TMEFF2-F1 | tTttttTTttCGGACGtCGtT-fluo |
| Seq-ID 4 | TMEFF2-R1 | red640-tCGGtCGATGtTttCGGtAA-pho |
| Seq-ID 5 | TMEFF2- | AAaAaAAAaaCTCCTCTaCATACGCCGCGaaTaaaTTaCCGaaAaCATCGaCCG |
| | amplicon | aaCAaCGaCGTCCGaaAAaaaaAaAaCGaaCTCCATTTaTTaaCCCAaaCAaTaAC |

fluo = fluoresceine label, red640 = LightCycler fluorescence label for channel F2, pho = 3′-OH-Phosphorylation.
Small written t's represent cytosines that were converted by bisulfite treatment, small a's represent complementary adenosine bases in the reverse complement synthesized strand.
Italic type letters in the primer sequences represent the added Gsu I recognition sites, which add generic ends to the PCR products.

TABLE 2
Results of the bisulfite specific real-time PCR for the TMEFF2 gene.

| | | | Cp of 2 replicates | Cp of 2 replicates |
|---|---|---|---|---|
| | | | of reactions | of reactions |
| DNA | Template DNA | | without Gsu I | with 1 unit Gsu I |
| TMEFF2 PCR products | 10⁴ | copies | 27.24 | 41.60 |
| with generic Gsu I sites | | | 27.56 | 40.80 |
| TMEFF2 PCR products | 10³ | copies | 32.71 | — |
| generic Gsu I sites | | | 32.81 | — |
| Bisulfite treated DNA | 1.0 | ng | 30.98 | 30.00 |
| (universal methylated) | | | 30.71 | 30.85 |

Collected are the crossing points (Cp) measured for 1 ng bisulfite-converted template DNA and the re-amplification of 10³ and 10⁴ copies of PCR product. Values for the method according to the present invention and for a control experiment, in which Gsu I was used, are shown.

TABLE 3
Type II restriction enzymes cleaving outside their recognition sequences

| | | Reach on | | | |
|---|---|---|---|---|---|
| | Reach on | bottom | | | |
| Seq-ID No | top strand | strand | Enzyme | Recognition Sequence | Isoschizomers |
| Seq-ID 6 | 20 | 18 | MmeI | TCCRACNNNNNNNNNNNNNNNNNNNN\hat{ } | — |
| | | | | \hat{ }NNNNNNNNNNNNNNNNNNGTYGGA | |
| Seq-ID 7 | 20 | 18 | CstMI | AAGGAGNNNNNNNNNNNNNNNNNNNN\hat{ } | — |
| | | | | \hat{ }NNNNNNNNNNNNNNNNNNCTCCTT | |
| Seq-ID 8 | 16 | 14 | GsuI | CTGGAGNNNNNNNNNNNNNNNN\hat{ } | BpmI |
| | | | | \hat{ }NNNNNNNNNNNNNNCTCCAG | |
| Seq-ID 9 | 16 | 14 | Eco57MI | CTGRAGNNNNNNNNNNNNNNNN\hat{ } | — |
| | | | | \hat{ }NNNNNNNNNNNNNNCTYCAG | |
| Seq-ID 10 | 16 | 14 | Eco57I | CTGAAGNNNNNNNNNNNNNNNN\hat{ } | AcuI BspKT5I |
| | | | | \hat{ }NNNNNNNNNNNNNNCTTCAG | |
| Seq-ID 11 | 16 | 14 | BsgI | GTGCAGNNNNNNNNNNNNNNNN\hat{ } | — |
| | | | | \hat{ }NNNNNNNNNNNNNNCTGCAC | |
| Seq-ID 12 | 16 | 14 | Bce83I | CTTGAGNNNNNNNNNNNNNNNN\hat{ } | BpuEI |
| | | | | \hat{ }NNNNNNNNNNNNNNCTCAAG | |
| Seq-ID 13 | 15 | 9 | CjeI | CCANNNNNNGTNNNNNNNNNNNNNNN\hat{ } | — |
| | | | | \hat{ }NNNNNNNNNACNNNNNNTGG | |
| Seq-ID 14 | 15 | 10 | BaeI | GRTACNNNNGTNNNNNNNNNNNNNNN\hat{ } | — |
| | | | | \hat{ }NNNNNNNNNNACNNNNGTAYC | |
| Seq-ID 15 | 14 | 9 | HaeIV | GAYNNNNNRTCNNNNNNNNNNNNNN\hat{ } | — |
| | | | | \hat{ }NNNNNNNNNGAYNNNNNRTC | |
| Seq-ID 16 | 14 | 8 | CjePI | CCANNNNNNNTCNNNNNNNNNNNNNN\hat{ } | — |
| | | | | \hat{ }NNNNNNNNGANNNNNNNTGG | |
| Seq-ID 17 | 14 | 8 | CjeI | ACNNNNNNTGGNNNNNNNNNNNNNN\hat{ } | — |
| | | | | \hat{ }NNNNNNNNCCANNNNNNGT | |
| Seq-ID 18 | 13 | 8 | TstI | GGANNNNNNGTGNNNNNNNNNNNNN\hat{ } | — |
| | | | | \hat{ }NNNNNNNNCACNNNNNNTCC | |
| Seq-ID 19 | 13 | 8 | PpiI | GAACNNNNNCTCNNNNNNNNNNNNN\hat{ } | — |
| | | | | \hat{ }NNNNNNNNGAGNNNNNGTTC | |
| Seq-ID 20 | 13 | 8 | Hin4I | GABNNNNNRTCNNNNNNNNNNNNN\hat{ } | — |
| | | | | \hat{ }NNNNNNNNGAYNNNNNVTC | |
| Seq-ID 21 | 13 | 8 | Hin4I | GAYNNNNNVTCNNNNNNNNNNNNN\hat{ } | — |
| | | | | \hat{ }NNNNNNNNGABNNNNNRTC | |
| Seq-ID 22 | 13 | 7 | HaeIV | GAYNNNNNRTCNNNNNNNNNNNNN\hat{ } | — |
| | | | | \hat{ }NNNNNNNGAYNNNNNRTC | |
| Seq-ID 23 | 13 | 8 | FalI | AAGNNNNNCTTNNNNNNNNNNNNN\hat{ } | — |
| | | | | \hat{ }NNNNNNNNAAGNNNNNCTT | |
| Seq-ID 24 | 13 | 11 | CspCI | CCACNNNNNTTGNNNNNNNNNNNNN\hat{ } | — |
| | | | | \hat{ }NNNNNNNNNNNCAANNNNNGTGG | |
| Seq-ID 25 | 13 | 7 | CjePI | GANNNNNNNTGGNNNNNNNNNNNNN\hat{ } | — |
| | | | | \hat{ }NNNNNNNCCANNNNNNNTC | |
| Seq-ID 26 | 13 | 8 | Bsp24I | CCANNNNNNGTCNNNNNNNNNNNNN\hat{ } | — |
| | | | | \hat{ }NNNNNNNNGACNNNNNNTGG | |
| Seq-ID 27 | 13 | 8 | BplI | GAGNNNNNCTCNNNNNNNNNNNNN\hat{ } | — |
| | | | | \hat{ }NNNNNNNNGAGNNNNNCTC | |
| Seq-ID 28 | 12 | 7 | TstI | CACNNNNNNTCCNNNNNNNNNNNN\hat{ } | — |
| | | | | \hat{ }NNNNNNNGGANNNNNNGTG | |
| Seq-ID 29 | 12 | 9 | RleAI | CCCACANNNNNNNNNNNN\hat{ } | — |
| | | | | \hat{ }NNNNNNNNNTGTGGG | |
| Seq-ID 30 | 12 | 7 | PsrI | GTANNNNNNGTTCNNNNNNNNNNNN\hat{ } | — |
| | | | | \hat{ }NNNNNNNGAACNNNNNNTAC | |
| Seq-ID 31 | 12 | 7 | PsrI | GAACNNNNNNTACNNNNNNNNNNNN\hat{ } | — |
| | | | | \hat{ }NNNNNNNGTANNNNNNGTTC | |
| Seq-ID 32 | 12 | 7 | PpiI | GAGNNNNNGTTCNNNNNNNNNNNN\hat{ } | — |
| | | | | \hat{ }NNNNNNNGAACNNNNNCTC | |
| Seq-ID 33 | 12 | 10 | CspCI | CAANNNNNGTGGNNNNNNNNNNNN\hat{ } | — |
| | | | | \hat{ }NNNNNNNNNNCCACNNNNNTTG | |
| Seq-ID 34 | 12 | 7 | Bsp24I | GACNNNNNNTGGNNNNNNNNNNNN\hat{ } | — |
| | | | | \hat{ }NNNNNNNCCANNNNNNGTC | |
| Seq-ID 35 | 12 | 9 | BsaXI | GGAGNNNNNGTNNNNNNNNNNNN\hat{ } | — |
| | | | | \hat{ }NNNNNNNNNACNNNNNCTCC | |
| Seq-ID 36 | 12 | 10 | BdaI | TGANNNNNNTCANNNNNNNNNNNN\hat{ } | — |
| | | | | \hat{ }NNNNNNNNNNTGANNNNNNTCA | |
| Seq-ID 37 | 12 | 10 | BcgI | GCANNNNNNTCGNNNNNNNNNNNN\hat{ } | — |
| | | | | \hat{ }NNNNNNNNNNCGANNNNNNTGC | |
| Seq-ID 38 | 12 | 10 | BcgI | CGANNNNNNTGCNNNNNNNNNNNN\hat{ } | — |
| | | | | \hat{ }NNNNNNNNNNGCANNNNNNTCG | |
| Seq-ID 39 | 12 | 13 | BcefI | ACGGCNNNNNNNNNNNN\hat{ } | — |
| | | | | \hat{ }NNNNNNNNNNNNNGCCGT | |
| Seq-ID 40 | 12 | 14 | BceAI | ACGGCNNNNNNNNNNNN\hat{ } | — |
| | | | | \hat{ }NNNNNNNNNNNNNNGCCGT | |
| Seq-ID 41 | 12 | 7 | BaeI | ACNNNNGTAYCNNNNNNNNNNNN\hat{ } | — |
| | | | | \hat{ }NNNNNNNGRTACNNNNGT | |
| Seq-ID 42 | 12 | 7 | AloI | GGANNNNNNGTTCNNNNNNNNNNNN\hat{ } | — |
| | | | | \hat{ }NNNNNNNGAACNNNNNNTCC | |
| Seq-ID 43 | 12 | 7 | AloI | GAACNNNNNNTCCNNNNNNNNNNNN\hat{ } | — |
| | | | | \hat{ }NNNNNNNGGANNNNNNGTTC | |
| Seq-ID 44 | 12 | 10 | AlfI | GCANNNNNNTGCNNNNNNNNNNNN\hat{ } | — |
| | | | | \hat{ }NNNNNNNNNNGCANNNNNNTGC | |
| Seq-ID 45 | 12 | 10 | AlfI | GCANNNNNNTGCNNNNNNNNNNNN\hat{ } | — |
| | | | | \hat{ }NNNNNNNNNNGCANNNNNNTGC | |
| Seq-ID 46 | 12 | 7 | AjuI | CCAANNNNNNNTTCNNNNNNNNNNNN\hat{ } | — |
| | | | | \hat{ }NNNNNNNGAANNNNNNNTTGG | |
| Seq-ID 47 | 11 | 9 | Tth11III | CAARCANNNNNNNNNNN\hat{ } | — |
| | | | | \hat{ }NNNNNNNNNTGYTTG | |
| Seq-ID 48 | 11 | 9 | TspGWI | ACGGANNNNNNNNNNN\hat{ } | — |
| | | | | \hat{ }NNNNNNNNNTCCGT | |
| Seq-ID 49 | 11 | 9 | TspDTI | ATGAANNNNNNNNNNN\hat{ } | — |
| | | | | \hat{ }NNNNNNNNNTTCAT | |
| Seq-ID 50 | 11 | 9 | TsoI | TARCCANNNNNNNNNNN\hat{ } | — |
| | | | | \hat{ }NNNNNNNNNTGGYTA | |
| Seq-ID 51 | 11 | 9 | TaqII | CACCCANNNNNNNNNNN\hat{ } | — |
| | | | | \hat{ }NNNNNNNNNTGGGTG | |
| Seq-ID 52 | 11 | 9 | TaqII | GACCGANNNNNNNNNNN\hat{ } | — |
| | | | | \hat{ }NNNNNNNNNTCGGTC | |
| Seq-ID 53 | 11 | 9 | EciI | GGCGGANNNNNNNNNNN\hat{ } | — |
| | | | | \hat{ }NNNNNNNNNTCCGCC | |
| Seq-ID 54 | 11 | 6 | AjuI | GAANNNNNNNTTGGNNNNNNNNNNN\hat{ } | — |
| | | | | \hat{ }NNNNNNCCAANNNNNNNTTC | |
| Seq-ID 55 | 10 | 14 | StsI | GGATGNNNNNNNNNN\hat{ } | — |
| | | | | \hat{ }NNNNNNNNNNNNNNCATCC | |
| Seq-ID 56 | 10 | 14 | BtgZI | GCGATGNNNNNNNNNN\hat{ } | — |
| | | | | \hat{ }NNNNNNNNNNNNNNCATCGC | |
| Seq-ID 57 | 10 | 14 | BsmFI | GGGACNNNNNNNNNN\hat{ } | BpuSI BslFI |
| | | | | \hat{ }NNNNNNNNNNNNNNGTCCC | BspLU11III |
| | | | | | BstOZ616I FaqI |
| Seq-ID 58 | 10 | 8 | BseRI | GAGGAGNNNNNNNNNN\hat{ } | — |
| | | | | \hat{ }NNNNNNNNCTCCTC | |
| Seq-ID 59 | 10 | 8 | BseMII | CTCAGNNNNNNNNNN\hat{ } | — |
| | | | | \hat{ }NNNNNNNNCTGAG | |
| Seq-ID 60 | 10 | 7 | BsaXI | ACNNNNNCTCCNNNNNNNNNN\hat{ } | — |
| | | | | \hat{ }NNNNNNNGGAGNNNNNGT | |
| Seq-ID 61 | 9 | 13 | FokI | GGATGNNNNNNNNN\hat{ } | — |
| | | | | \hat{ }NNNNNNNNNNNNNCATCC | |
| Seq-ID 62 | 9 | 7 | BspCNI | CTCAGNNNNNNNNN\hat{ } | — |
| | | | | \hat{ }NNNNNNNCTGAG | |
| Seq-ID 63 | 8 | 8 | SspD5I | GGTGANNNNNNNN\hat{ } | — |
| | | | | \hat{ }NNNNNNNNTCACC | |
| Seq-ID 64 | 8 | 7 | MboII | GAAGANNNNNNNN\hat{ } | NcuI |
| | | | | \hat{ }NNNNNNNTCTTC | |
| Seq-ID 65 | 8 | 7 | HphI | GGTGANNNNNNNN\hat{ } | AsuHPI |
| | | | | \hat{ }NNNNNNNTCACC | |
| Seq-ID 66 | 8 | 12 | BbvI | GCAGCNNNNNNNN\hat{ } | AlwXI BseKI |
| | | | | \hat{ }NNNNNNNNNNNNGCTGC | BseXI Bsp423I |
| | | | | | Bst12I Bst71I |
| | | | | | BstVII |
| Seq-ID 67 | 7 | 6 | MnII | CCTCNNNNNNN\hat{ } | — |
| | | | | \hat{ }NNNNNNGAGG | |
| Seq-ID 68 | 7 | 11 | Bbr7I | GAAGACNNNNNNN\hat{ } | — |
| | | | | \hat{ }NNNNNNNNNNNGTCTTC | |
| Seq-ID 69 | 7 | 11 | AceIII | CAGCTCNNNNNNN\hat{ } | — |
| | | | | \hat{ }NNNNNNNNNNNGAGCTG | |

### Example 2

### Methylation Analysis of the TMEFF2 Gene in Colorectal Cancer and Normal Tissue Using a Primer Comprising a Generic Gsu I-Recognition Site

In this example bisulfite converted DNA from tissue was amplified using primers comprising a Gsu I recognition site. Thereby, the methylated DNA of the TMEFF2 gene was amplified (as described in example 1). Previously generated PCR products were added to these reactions to simulate a carry-over cross contamination. By preincubation of the PCR reaction with 1 unit Gsu I endonuclease, the PCR products were efficiently inactivated, so that the methylation analysis results were similar to the setup without any contaminating PCR products. To achieve this, the following steps were carried out:

1 μg of genomic DNA (200 μl) was extracted from tumors and normal adjacent tissue of 12 patients with colon cancer, respectively. The 24 samples obtained were treated as described above and 10 μl were used as template in a PCR reaction.

As reference DNA for quantification of sample derived total and methylated DNA, universally methylated human DNA was used. 1 μg GpGenome™ Universal Methylated DNA (Chemicon International, Temecula USA) was bisulfite converted, purified and quantified by measuring the extinction at 260 nm. The bisulfite treatment was performed as previously described (see for example WO2005038051). Subsequently the DNA was adjusted to 12.5 ng, 2.5 ng, 0.5 ng and 0.1 ng per 10 μl. This standard DNA dilution series was stored at 4° C. for several hours and 10 μl were used as template in a PCR reaction.

For the quantification of the total DNA of the samples, the C3 reference PCR was used as previously described (EP05075404). A 20 μl reaction mixture contained:
10 μl of template DNA
2 μl of FastStart LightCycler Mix for hybridization probes (Roche Diagnostics)
3.5 mmol/l MgCl2 (Roche Diagnostics)
0.60 μmol/l forward primer (Seq ID-75, TIB-MolBiol)
0.60 μmol/l reverse primer (Seq ID-76, TIB-MolBiol)
0.2 μmol/l probel (Seq ID-77, TIB-MolBiol)

Sequences of oligonucleotides used in this example are listed in table 4.

The assay was performed in the LightCycler 2.0 according to the following temperature-time-profile:
Activation 10 min at 95° C.
50 cycles: 10 sec at 95° C.
   30 sec at 56° C.
   10 sec at 72° C.

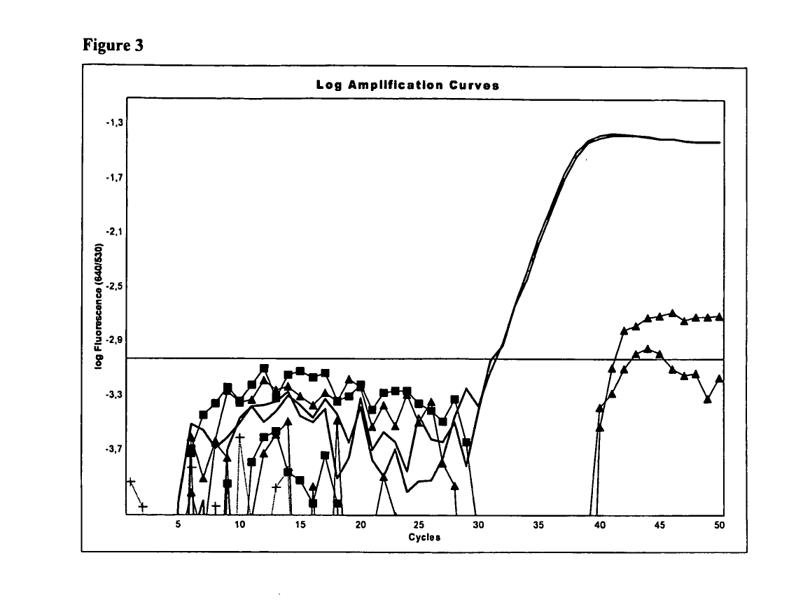

The primers used (Seq ID-75 and Seq ID-76) amplify a fragment of 130 by of the GSTP1 gene (Seq ID-78; nucleotide 2273 to nucleotide 2402 of GenBank Accession Number X08058). The detection was carried out during the annealing phase at 56° C. in channel F1 at 530 nm. The crossing points (Cp) were calculated according to the “second derivative maximum” method by means of the LightCycler software. Representative amplification curves are shown in FIG. 3.

The carry-over prevention system according to the invention was then performed in the methylation specific TMEFF2 real-time PCR reaction (as described in example 1) by adding 1 unit Gsu I/PCR to the reaction mix, combined with a preincubation of the closed reaction vessel for 30 min at 30° C. The real-time PCR was performed in a LightCycler instrument and the crossing points (Cp) were determined by the automated LightCycler software using the “second derivative maximum” method.

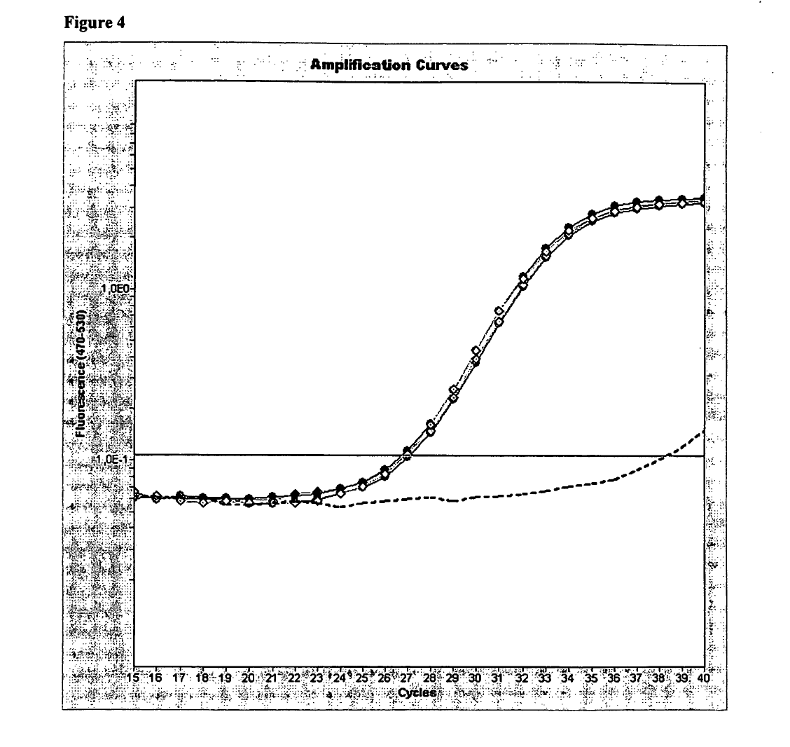

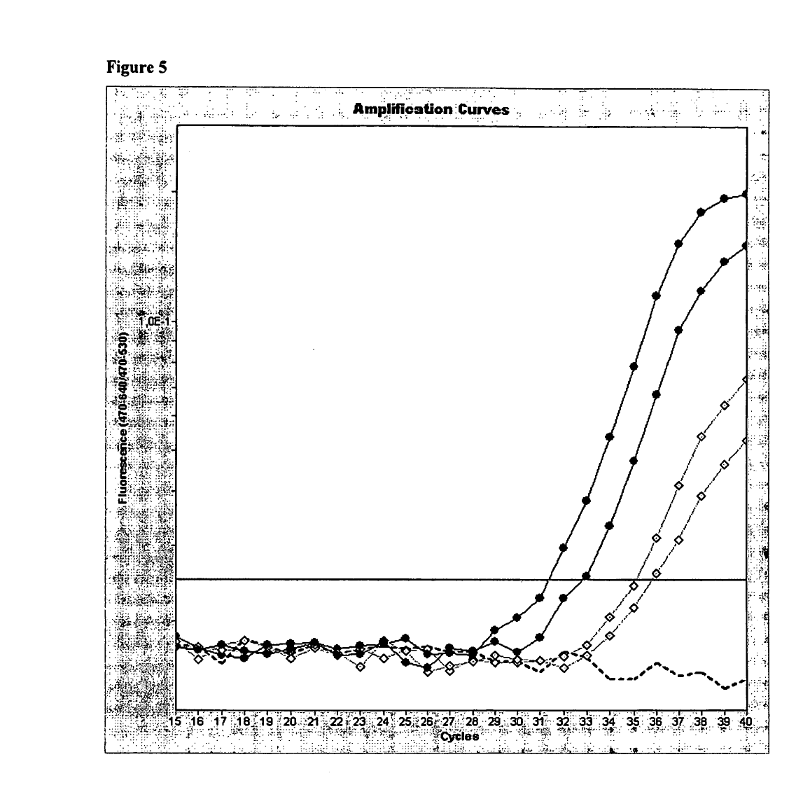

In the first round of methylation analysis, the reactions contained 1 unit/PCR Gsu I and TMEFF2 PCR product from methylated DNA. The addition of these PCR products simulated the case of a PCR cross contamination. Representative amplification curves are shown in FIG. 4. In a second round, the samples were analyzed in reaction mixes without Gsu I, which were also not contaminated by PCR products.

For the generation of PCR products, 10 ng methylated bisulfate converted DNA were amplified by means of the HeavyMethyl assay for TMEFF2. The PCR products were purified with the QIAquick PCR Purification Kit (Quiagen) and subsequently analyzed on a 2% agarose gel. After this, a serial dilution with water was carried out to a final dilution of 1:3×10¹⁰. 2 μl of this dilution were reamplified and quantified according to the Heavy-Methyl PCR for TMEFF2. The copy number was determined: 2 μl of the said dilution contain 3,000 copies of PCR product and were added to reactions of the first round of the methylation analysis.

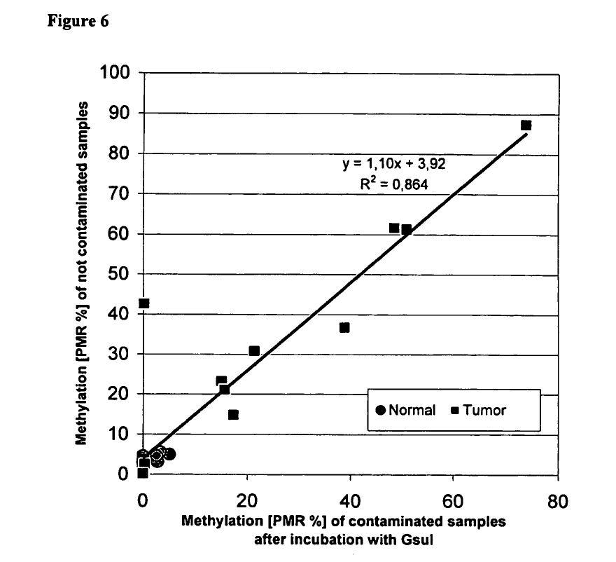

The methylation ratios were calculated according to the PMR value method (Eads et al., Cancer Res. 2001 Apr. 15; 61(8): 3410-8, PMID 11309301) against the total DNA quantified by the C3 reference PCR. The PMR values were calculated separately from the PCR results of round one and two and are summarized in table 5. The results confirmed the efficiency of PCR product inactivation by Gsu I endonuclease, because the methylation values were not significantly influenced by the addition of methylated PCR products. FIG. 6 shows the correlation between the measurements of the contaminated reactions and the reaction, which were not spiked with PCR product. The coefficient of determination (R-squared) was determined to be 0.864. This demonstrates the usefulness of the present method to prevent cross contamination of amplification reactions by PCR products.

TABLE 4
Sequences of oligonucleotides used in example 2

| Seq ID No | Name | Sequence |
|---|---|---|
| Seq-ID 70 | Gsu-TMEFF2 | 5′-CTGGAGCTGGAGAAaAaAAAaaCTCCTCTaCATAC |
| Seq-ID 71 | Gsu-TMEFF2 | 5′-CTGGAGCTGGAGGTtAtTGttTGGGttAAtAAATG |
| Seq-ID 72 | TMEFF2-F1 | tTttttTTttCGGACGtCGtT-fluo |
| Seq-ID 73 | TMEFF2-R1 | red640-tCGGtCGATGtTttCGGtAA-pho |
| Seq-ID 74 | TMEFF2- | AAaAaAAAaaCTCCTCTaCATACGCCGCGaaTaaaTTaCCGaaAaCATCGaCCG |
| | amplicon | aaCAaCGaCGTCCGaaAAaaaaAaAaCGaaCTCCATTTaTTaaCCCAaaCAaTaAC |
| Seq-ID 75 | C3F | GGAGTGGAGGAAAtTGAGAt |
| Seq-ID 76 | C3R | CCACACAaCAaaTaCTCAaAaC |
| Seq-ID 77 | C3-TAQ | FAM-TGGGTGTTTGTAATTTTTGTTTTGTGTTAGGTT-BHQ1 |
| Seq-ID 78 | C3-amplicon | GGAGTGGAGGAAAtTGAGAtttAtTGAGGTTACGTAGITTGtttAAGGTtAAG |
| | | ttTGGGTGttTGtAATttTTGtttTGTGttAGGtTGttTtttAGGIGTtAGGTGAGtTtTG |
| | | AGtAttTGtTGTGTGG |

fluo = fluorescein label, red640 = LightCycler fluorescence label for channel F2, pho = 3′-OH-Phosphorylation.
Small written t's represent cytosines that were converted by bisulfite treatment, small a's represent complementary adenosine bases in the reverse complement synthesized strand.
Italic type letters in the primer sequences represent the added Gsu I recognition sites, which add generic ends to the PCR product.

TABLE 5
Results of a methylation analysis of the TMEFF2
gene from colorectal cancer and from normal tissue,
obtained by two different methods.

| | | C | D |
|---|---|---|---|
| A | B | PMR [%] from | PMR [%] from not |
| Sample | Sample | contaminated samples | contaminated samples |
| name | type | measured with Gsu I | measured without Gsu I |
| 2NT | normal | 5.1 | 4.9 |
| 2TU | tumor | 0.1 | 0.0 |
| 3NT | normal | 0.3 | 2.3 |
| 3TU | tumor | 21.2 | 30.8 |
| 5NT | normal | 0.0 | 4.5 |
| 5TU | tumor | 48.4 | 61.7 |
| 7NT | normal | 0.0 | 3.5 |
| 7TU | tumor | 0.1 | 42.6 |
| 8NT | normal | 2.8 | 3.1 |
| 8TU | tumor | 15.0 | 23.2 |
| 9NT | normal | 0.1 | 3.1 |
| 9TU | tumor | 0.3 | 2.6 |
| 10NT | normal | 2.6 | 5.0 |
| 10TU | tumor | 15.6 | 21.1 |
| 12NT | normal | 0.1 | 1.1 |
| 12TU | tumor | 50.8 | 61.3 |
| 17NT | normal | 0.3 | 2.7 |
| 17TU | tumor | 73.7 | 87.4 |
| 18NT | normal | 3.3 | 5.6 |
| 18TU | tumor | 38.8 | 36.7 |
| 21NT | normal | 0.0 | 0.2 |
| 21TU | tumor | 0.0 | 0.2 |
| 24NT | normal | 2.6 | 4.6 |
| 24TU | tumor | 17.3 | 14.8 |

The PMR values were calculated from the proportion of the methylated DNA of the TMEFF2 gene according to the TMEFF2 HeavyMethyl assay against the total DNA obtained with the reference PCR. The values in column C are the results from samples spiked with 3,000 copies of TMEFF2 PCR product, whereas the reaction mix contained 1 unit Gsu I endonuclease. The values in column D were calculated from reactions without Gsu I, in which the samples were not contaminated with PCR products

1. A method for providing a decontaminated template nucleic acid for enzyme-mediated amplification reactions suitable for DNA methylation analysis, characterized by
a) incubating a nucleic acid with a chemical reagent or an enzyme containing solution whereby the unmethylated cytosine bases are converted into uracil bases, and a template nucleic acid is provided
b) mixing the template nucleic acid from step a) with the components required for an enzymatic amplification reaction, including
at least two oligonucleotides, whereby at least one of said oligonucleotides comprises
i) at least one sequence part that hybridizes with a sequence of the template nucleic acid to be amplified, and
ii) at least one sequence part that constitutes a recognition site for a DNA cleaving enzyme that cleaves DNA downstream of said recognition site and
c) adding to this mixture a DNA cleaving enzyme, which specifically binds to the at least one sequence part that is a recognition site, and
d) incubating the mixture, whereby nucleic acids containing said recognition site for a DNA cleaving enzyme are degraded.

2. The method of claim 1, with the subsequent steps of
e) incubating the mixture at an increased temperature, whereby the enzymatic DNA cleaving activity is terminated, and
f) amplifying the template nucleic acid.

3. The method according to claim 1, wherein the at least one of said oligonucleotides comprises several, preferably two to four, sequence parts that each constitutes a recognition site for a DNA cleaving enzyme.

4. The method according to claim 1, wherein the sequence parts that each constitute a recognition site for a DNA cleaving enzyme form a tandem repeat.

5. The method according to claim 1, wherein the at least one sequence part that hybridizes with a sequence of the template nucleic acid to be amplified of the at least one of said oligonucleotides comprises at least three nucleotides that hybridize to nucleotides on the template DNA which have been converted from a cytosine base to a uracil base.

6. (canceled)

7. (canceled)

8. (canceled)

9. The method according to claim 1, wherein the DNA cleaving enzyme cleaves the DNA at least 10 nucleotides, preferably at least 15 nucleotides, and most preferably at least 20 nucleotides downstream from its recognition site.

10. (canceled)

11. (canceled)

12. (canceled)

13. (canceled)

14. (canceled)

15. (canceled)

16. Method for providing a decontaminated template nucleic acid for enzymatic amplification reactions, characterized by
a) mixing a template nucleic acid with the components required for an amplification mediated by at least one ligase or based on transcription, including at least two oligonucleotides, whereby at least one of said oligonucleotides comprises at least one sequence part that hybridizes with a sequence of the template nucleic acid to be amplified, and at least one sequence part that constitutes a recognition site for a DNA cleaving enzyme that cleaves DNA downstream of said recognition site and
b) adding to this mixture at least one DNA cleaving enzyme, which specifically binds to the at least one sequence part that is a recognition site, and incubating the mixture, whereby nucleic acids containing said recognition site for a DNA cleaving enzyme are degraded.

17. Method according the to claim 16, with the subsequent steps of:
c) incubating the mixture at an increased temperature, whereby the enzymatic DNA cleaving activity is terminated, and
d) amplifying the template nucleic acid.

18. Test kit which can be used for the realization of the method according to any of claim 1, 2, 3, 4, 5, 9, 16 or 17, with the following components:
a) a reagent or an enzyme which converts unmethylated cytosines into uracil
b) an enzymatic activity, which specifically binds to a recognition site and cleaves DNA downstream from the binding site, and
c) at least one oligonucleotide which comprises:
i) at least one sequence part that hybridizes with a sequence of the template nucleic acid to be amplified, and
ii) at least one sequence part that constitutes a recognition site for a DNA cleaving enzyme that cleaves DNA downstream of said recognition site.

19. (canceled)

20. (canceled)

21. (canceled)

22. (canceled)

23. (canceled)

24. Use of an enzyme listed in table 3 for the generation of contamination free nucleic acids for methylation analysis, in particular according to the method of any of claim 1, 2, 3, 4, 5, 9, 16 or 17.

25. (canceled)

26. An oligonucleotide, particularly a primer oligonucleotide, which comprises:
i) at least one sequence part that hybridizes with a sequence of the template nucleic acid to be amplified, and
ii) at least one sequence part that constitutes a recognition site for a DNA cleaving enzyme that cleaves DNA downstream of said recognition site,
iii) wherein the sequence part that hybridizes with the sequence to be amplified only contains either the nucleotides C, T, and A, or G, T, and A.
